(19) United States
(12) Patent Application Publication
(10) Pub. No.: US 2021/0252173 A1
(43) Pub. Date: Aug. 19, 2021

(54) STABILIZED POLYUNSATURATED COMPOUNDS AND USES THEREOF

(71) Applicant: RETROTOPE, INC., Los Altos, CA (US)

(72) Inventors: Mikhail S. SHCHEPINOV, Oxford (GB); Robert J. MOLINARI, Los Altos, CA (US); Peter MILNER, Los Altos, CA (US)

(73) Assignee: Retrotope, Inc., Los Altos, CA (US)

(21) Appl. No.: 17/049,017

(22) PCT Filed: Apr. 18, 2019
(86) PCT No.: PCT/US2019/028081

§ 371 Date: Oct. 19, 2020

Related U.S. Application Data
(60) Provisional application No. 62/660,843, filed on Apr. 20, 2018.
(60) Provisional application No. 62/660,823, filed on Apr. 20, 2018.

(51) Int. Cl. A61K 51/04; A61K 45/06; A61K 9/00; A61P 25/28

(52) U.S. Cl. CPC A61K 51/0402; A61K 45/06; C07B 2200/05; A61P 25/28; A61K 9/0053

(57) ABSTRACT

Methods are provided for treating a subject having, or at risk of, a lysosomal storage disease, particularly Tay-Sachs, Gaucher disease, Sandhoff disease or Niemann-Pick disease, neuronal ceroid lipofuscinosis, or a condition associated with impaired Phospholipase A2 Group VI (PLA2G6) activity, particularly infantile neuroaxonal dystrophy or PLA2G6 associated neurodegeneration (PLAN), or a sleeping disorder, using a substituted polyunsaturated fatty acid, polyunsaturated fatty acid ester, polyunsaturated fatty acid thioester, fatty acid amide, polyunsaturated fatty acid mimetic, polyunsaturated fatty acid pro-drug, or combinations thereof, where the substituted compound comprises at least one substitution that reduces oxidation of the compound. Preferably, the substituted compound is a deuterated polyunsaturated fatty acid, or an ethyl ester thereof, such as 11,11-D2-linoleic acid, 11,11-D2-linoleic acid ethyl ester, 11,11,14,14-D4-linolenic acid, or 11,11,14,14-D4-linolenic acid ethyl ester.

### BACKGROUND

### Field

The present disclosure relates to the fields of biochemistry and chemistry. Some embodiments relate to stabilized polyunsaturated substances, composition comprising such stabilized polyunsaturated substances, and the therapeutic use thereof.

### Description of the Related Art

In biological systems, the formation of potentially physiologically-deleterious reactive oxygen species (ROS) and reactive nitrogen species (RNS), may be caused by a variety of metabolic and/or environmental processes. By way of non-limiting example, intracellular ROS (e.g., hydrogen peroxide H₂O₂; superoxide anion O₂⁻; hydroxyl radical OH⁻; nitric oxide NO; and the like) may be generated by several mechanisms: (i) by the activity of radiation, both exciting (e.g., UV-rays) and ionizing (e.g., X-rays); (ii) during xenobiotic and drag metabolism; and (iii) under relatively hypoxic, ischemic and catabolic metabolic conditions, as well as by exposure to hyperbaric oxygen. Protection against the harmful physiological activity of ROS and RNS species is mediated by a complex network of overlapping mechanisms and metabolic pathways that utilize a combination of small redox-active molecules and enzymes coupled with the expenditure of reducing equivalents.

Concentrations of ROS and RNS which cannot be adequately dealt with by the endogenous antioxidant system can lead to damage of lipids, proteins, carbohydrates, and nucleic acids. Changes in oxidative metabolism which lead to an increase in the oxidizing environment and the formation of potentially physiologically-deleterious ROS and RNS have been generally termed within the literature as “oxidative stress.” There is a need for an effective method to inhibit or reduce such oxidative stress and further treatment of medical conditions associated with oxidative stress.

### SUMMARY

Some embodiments relate to method of treating a subject having, or at risk for, a disease or condition associated with an impaired Phospholipase A2 Group VI (PLA2G6) activity, comprising administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drag, or combinations thereof, the substituted compound comprises at least one substitution that reduces oxidation of the substituted compound.

Some embodiments relate to method of treating a subject having, or at risk for an infantile neuroaxonal dystrophy (INAD) or PLA2G6 associated neurodegeneration (PLAN), comprising administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drag, or combinations thereof, the substituted compound comprises at least one substituent that reduces oxidation of the substituted compound.

Some embodiments relate to method of treating a subject having, or at risk for, a disease or condition associated with a lysosomal storage disease (LSD), comprising administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, or combinations thereof, the substituted compound comprising at least one substituent that reduces oxidation of the substituted compound.

Some embodiments relate to method of treating a subject having, or at risk for neuronal ceroid lipofuscinosis (NCL) type disease, comprising: administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, or a combination thereof, the substituted compound comprising at least one substituent that reduces oxidation of the substituted compound.

Some embodiments relate to method of treating a subject having, or at risk for, a sleeping disorder, comprising administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, or combinations thereof, the substituted compound comprising at least one substituent that reduces oxidation of the substituted compound.

### BRIEF DESCRIPTION OF THE DRAWINGS

FIG. 1 summarizes the baseline and one year treatment status of a patient described in Example 1 (degree of impairment: (0) for severely impaired, (+1) for moderately impaired, and (+2) for mildly impaired or no impairment).

### DETAILED DESCRIPTION

Lipid peroxidation (LPO) is a self-propagating, free-radical chain reaction that amplifies toxic triggering effects in a variety of neurodegenerative conditions. The present disclosure relates to method of treating various diseases and conditions using isotopically modified polyunsaturated fatty acids or derivatives thereof, where these isotopically modified compounds have been stabilized via isotopic substitution at least one position that reduces oxidation of the compounds. These substituted compounds may be readily incorporated into cell membranes and may prevent, delays, or reverse lipid peroxidation and the oxidative damage caused by LPO.

Diseases Associated with PLA2G6

The PLA2G6 gene encodes a group VIA calcium-independent phospholipase A2 beta enzyme that selectively hydrolyses glycerophospholipids to release free fatty acids. Mutations in PLA2G6 have been associated with disorders such as infantile neuroaxonal dystrophy (INAD), neurodegeneration with brain iron accumulation type II and Karak syndrome. PLA2G6 can be the causative gene in a subgroup of patients with autosomal recessive early-onset dystonia-parkinsonism. Neuropathological examination can show widespread Lewy body pathology and the accumulation of hyperphosphorylated tau.

In some studies, the mechanism of disease onset and progression indicates that lipid peroxidation pathways are the causes of disease phenotypes. In INAD model, PLA2G6 mutations trigger accumulation of lipid peroxidation products of linoleic acid and other PUFAs leading to PLA2G6 associated neurodegeneration (PLAN). Studies using a fly model of PLAN have been conducted to address whether indeed toxic oxidized cardiolipin is accumulating within the mitochondrial membranes. The knock-out led to significant elevation in lipid peroxidation within the fly brain due to buildup of intracellular accumulations of stored lipids. High concentrations of lipid peroxidation products were observed.

INAD is a neurodegenerative disease with onset in infancy and fatality in the teenage years or in early adulthood. It is characterized neuropathologically by axonal swelling and the presence of spheroid bodies in the central and peripheral nervous systems in addition to hallmark cerebellar atrophy. Neurodegeneration with brain iron accumulation comprises a clinically and genetically heterogeneous group of disorders with a progressive extrapyramidal syndrome and high basal ganglia iron, and includes pantothenate kinase-associated neurodegeneration caused by mutations in PANK2 (neurodegeneration with brain iron accumulation type I). Post-mortem examination of the brain of a patient with neurodegeneration with brain iron accumulation associated with homozygous PLA2G6 mutations have shown pathology with widespread Lewy bodies, dystrophic neurites and cortical neuronal neurofibrillary tangles.

Mutations in the PLA2G6 gene have been identified in most individuals with infantile neuroaxonal dystrophy. The PLA2G6 gene provides instructions for making an enzyme called an A2 phospholipase. This enzyme family is involved in metabolizing phospholipids. Phospholipid metabolism is important for many body processes, including helping to keep the cell membrane intact and functioning properly. The A2 phospholipase produced from the PLA2G6 gene, sometimes called PLA2 group VI, helps to regulate the levels of a compound called phosphatidylcholine, which is abundant in the cell membrane. Mutations in the PLA2G6 gene impair the function of the PLA2 group VI enzyme. This impairment of enzyme function may disrupt cell membrane maintenance and contribute to the development of spheroid bodies in the nerve axons. Phospholipid metabolism problems have been seen in both this disorder and a related disorder called pantothenate kinase-associated neurodegeneration.

### Lysosomal Storage Diseases

The term “lysosomal storage diseases” or “lysosomal storage disorders” (LSD) refers to a group of nearly fifty relatively rare inherited metabolic disorders that result from defects in lysosomal function as the result of deficiency of an enzyme, leading to the inappropriate storage of material in various cells of the body. These defects are related to deficient cellular metabolism of various types of lipids, glycoproteins and/or mucopolysaccharides. As a result of LSD, excess cell products that would ordinarily be broken down instead accumulate within the cell to an undesirable degree. Most lysosomal storage diseases are inherited in an autosomal recessive manner. The symptoms of lysosomal storage disorders are generally progressive over a period of time. Some exemplary lysosomal storage diseases include: Gaucher disease (Types I, II, and III), Pompe disease (glycogen storage disease, including infantile form and a delayed onset form), GM2 gangliosidosis (including Tay-Sachs disease and Sandhoff disease), GM1 gangliosidosis, and Niemann-Pick disease.

The term “neuronal ceroid lipofuscinosis” (NCL) is the general name for a family of genetically separate neurodegenerative lysosomal storage diseases that result from excessive accumulation of lipopigments (lipofuscin) in the body's tissues. These lipopigments are made up of fats and proteins. These lipofuscin materials build up in neuronal cells and many organs, including the liver, spleen, myocardium and kidneys.

GM1 gangliosidosis is a rare lysosomal storage disorder characterized biochemically by deficient beta-galactosidase activity and clinically by a wide range of variable neurovisceral, ophthalmological and dysmorphic features.

GM2 gangliosidoses are a group of three related genetic disorders that result from a deficiency of the enzyme beta-hexosaminidase. This enzyme catalyzes the biodegradation of fatty acid derivatives known as gangliosides. When beta-hexosaminidase is no longer functioning properly, the lipids accumulate in the nervous tissue of the brain and cause problems. GM2 gangliosidoses include Tay-Sachs disease, Sandhoff disease, and AB variant.

Tay-Sachs disease is a rare inherited disorder that progressively destroys nerve cells (neurons) in the brain and spinal cord. The most common form of Tay-Sachs disease becomes apparent in infancy. Other forms of Tay-Sachs disease are very rare. Signs and symptoms can appear in childhood, adolescence, or adulthood and are usually milder than those seen with the infantile form. Characteristic features include muscle weakness, loss of muscle coordination (ataxia) and other problems with movement, speech problems, and mental illness. These signs and symptoms vary widely among people with late-onset forms of Tay-Sachs disease. Tay-Sachs disease is caused by a genetic mutation in the HEXA gene on chromosome 15. The mutation results in problems with an enzyme called beta-hexosaminidase A which results in the buildup of the molecule GM2 ganglioside within cells, leading to toxicity. Diagnosis is by measuring the blood hexosaminidase A level or genetic testing. In some embodiments of the methods described herein, selecting for treatment a subject having Tay-Sachs disease includes measuring the blood hexosaminidase A level, or testing genetic mutation in the HEXA gene.

Sandhoff disease is a rare, autosomal recessive metabolic disorder that causes progressive destruction of nerve cells in the brain and spinal cord. The disease results from mutations on chromosome 5 in the HEXB gene, critical for the lysosomal enzymes beta-N-acetylhexosaminidase A and B. In some embodiments of the methods described herein, selecting for treatment a subject having Sandhoff disease includes testing genetic mutation in the HEXB gene.

Gaucher's disease (GD) is a genetic disorder in which glucocerebroside (a sphingolipid, also known as glucosylceramide) accumulates in cells and certain organs. The disorder is characterized by bruising, fatigue, anemia, low blood platelet count and enlargement of the liver and spleen, and is caused by a hereditary deficiency of the enzyme glucocerebrosidase (also known as glucosviceramidase), which acts on glucocerebroside. When the enzyme is defective, glucocerebroside accumulates, particularly in white blood cells and especially in macrophages (mononuclear leukocytes). Glucocerebroside can collect in the spleen, liver, kidneys, lungs, brain, and bone marrow. This disease is caused by a recessive mutation in the GBA gene located on chromosome 1, Gaucher's disease is the most common of the lysosomal storage diseases. It is a form of sphingolipidosis (a subgroup of lysosomal storage diseases), as it involves dysfunctional metabolism of sphingolipids. In some embodiments of the methods described herein, selecting for treatment a subject having Gaucher's disease includes testing genetic mutation in the GBA gene.

Niemann-Pick disease is are a subgroup of lysosomal storage disorders, which is a group of inherited, severe metabolic disorders in which sphingomyelin accumulates in lysosomes in ceils. Sphingomyelin is a component of cell membrane including the organellar membrane, so the enzyme deficiency blocks degradation of lipid, resulting in the accumulation of sphingomyelin within lysosomes in the macrophage-monocyte phagocyte lineage. Mutations in the SMPD1 gene cause Niemann-Pick disease types A and B. They produce a deficiency in the activity of the lysosomal enzyme acid sphingomyelinase, that breaks down the lipid sphingomyelin. Mutations in NPC1 or NPC2 cause Niemann-Pick disease, type C (NFC), which affects a protein used to transport lipids. For type A and B, levels of sphingomylinase can be measured from a blood sample. To diagnose type C, a skin sample can help determine whether the transporter is affected.

Typical drugs target enzymes, proteins, or gene pathways. However, many biochemical processes are not controlled by enzymes. These processes are not often addressed therapeutically, in part, because modern drug discovery is usually based on biochemical pathway mapping informed by genomic analysis, and such approaches may be relatively blind to non-genetically encoded events. Non-enzymatic in vivo processes include a large group of oxidation reactions. The resulting oxidative damage is detrimental and, in diseased cells, cannot be controlled by antioxidants. Antioxidants are typically present in cells at levels close to saturation through enzymatically controlled active transport, and their concentrations cannot be further increased easily. In addition, excessive levels of antioxidants may interfere with required redox processes and result in a net detrimental effect. This may explain why clinical trial s of antioxidants in humans often result in no positive or negative effects, even though the disease aetiology is oxidative in nature.

Lipid peroxidation may cause lysosomal instability and impairment of lysosome function, leading to LSD. As indicated above, LSD represents a class of inborn pathologies characterized by the accumulation of material in lysosomes. These conditions can be caused by the absence or reduced activity of lysosomal proteins, which results in the lysosomal accumulation of substances. Often, this material will be stored because digestion is impaired due to enzyme deficiency, but LSD can also arise when transport out of the lysosomal compartment is compromised. In some LSDs, the selection and transport of various structurally damaged moieties related to various lipid subclasses, for example sphingolipids, to lysosomes for processing can be compromised. Moreover, the accumulation of substances such as highly susceptible polyunsaturated fatty acids containing lipids can affect the function of lysosomes and, further downstream, other organelles, resulting in secondary changes, such as impairment of autophagy, mitochondrial dysfunction, and inflammation, LSDs frequently involve the central nervous system, where neuronal dysfunction or loss results in mental retardation, progressive motor degeneration, and premature death.

The reactive oxygen species (ROS) play a pivotal role and are perhaps common mediators of cell death in many LSDs. Thus, up-regulation of apurinic endonuclease 1 (APE1) (a protein that repairs oxidative DNA damage) has been observed in Gaucher fibroblasts (but not in Gaucher bone marrow mesenchymal stromal cells). In the GM1 and GM2 gangliosidoses, inducible nitric oxide synthase and nitrotyrosine are elevated in activated microglia/macro-phages, and ROS is elevated in Fabry disease models. Gene microarray analysis from the Niemann-Pick disease type C 1 (NPC1) fibroblasts is consistent with enhanced oxidative stress, and elevated ROS and lipid peroxidation renders the fibroblasts more susceptible to cell death after an acute oxidative insult. In mucopolysaccharidosis type IIIB (MPSIIIB), enhanced oxidative stress results in protein, lipid, and DNA oxidation, and an oxidative imbalance is found in mucopolysaccharidosis type I (MPSI). In neuronal ceroid lipofuscinose (NCL), elevated ROS and superoxide dismutase levels are suggested to be downstream to ER stress, a significant increase in manganese-dependent superoxide dismutase activity can be detected in fibroblasts and brain extracts from CLN6 sheep, and increased expression of 4-hydroxynonenal can be detected in late infant and juvenile forms of NCL.

The central role that oxidative stress plays in integrating other cellular pathways and stresses shows that it is most likely activated in LSDs as a secondary biochemical pathway, rather than as a direct result of accumulation of the primary substrate. Moreover, the possible role of oxidative stress may be of real significance in delineating LSD pathology, particularly as oxidative stress plays a central role in other better studied neurodegenerative conditions.

### Sleeping Disorders

A very large proportion (around 40%) of the adult population is affected by various aspects of dyssomnia, either chronic or acute. Sleep plays an important, multifunctional role in physiological homeostasis. Goel N. Sleep Med Clin 2011; 6:171. This includes the clean-up function, which sees to it that unwanted inter- and intra-neuronal metabolic products accumulated during the day are catabolized and removed. A significant fraction of such debris consists of oxidative stress generated materials such as various LPO derivatives, on their own or as conjugates with other biomolecules such a DNA, phospholipid head groups, proteins or peptides. Mathangi D C et al., Ann Neurosci 2012; 19:161; Thamaraiselvi K et al., Int. J. Biol. Med. Res. 2012; 3:1754. It is well recognized that such derivatives are elevated, and indeed can be used as markers, of insufficient sleep. Weljie A M et al., PNAS USA 2015; 112:2569.

External (lifestyle choices such as reduced sleep duration or jetlag) or internal (various sleeping disorders) factors adversely affect this elimination process, resulting in non-complete removal, or gradual accumulation, of LPO products, with ensuing metabolic pathologies, various neurological conditions, including, but not limited to, psychosis and bipolar disorder, as well as accelerated aging. Schmidt S M et al., Lancet Diabetes Endocrinol. 2014; 3:52. A related problem is disturbance of circadian rhythmicity and oscillation, which affects multiple metabolic pathways. Moeller-Level C S et al., PNAS USA 2013; 110:E1132. This is particularly relevant to lipid processing, which is controlled to a very large degree by circadian cycling. Chua E C-P et al., PNAS USA 2013; 110:14468.

Elevated oxidative stress markers are associated with obstructive sleep apnoea syndrome, and in general with many other subclasses of dyssomnia. Passali D. et al., Acta Otorhinolaryngol. Ital. 2015; 35:420; Haehul D E et al., Climacteric 2006; 9:312; Gulec M et al., Prog Neuropsychopharmacol Biol Psychiatry 2012; 37; 247; Liang B et al., Eur Rev Med Pharmacol Sci 2013; 17:2517; Semenova N V et al., Neuropsychiatry 2018; 8:1452.

In some embodiments, the substituted compounds such as D-PUFAs may be used either alone or in combination with other treatments (including but not limited to antioxidants, melatonin, glycine, sleep medication, antidepressants, etc.) to mitigate the side effects of insufficient sleep and sleep disorders caused by various background conditions, including but not limited to, lifestyle related sleep deficiency; alcohol related sleep deficiency; idiopathic hypersomnia; narcolepsy; various sleep apneas; various parasomnias; restless leg syndrome; sleep state misperception; chronic fatigue syndrome (CFS) (also referred to as myalgic encephalomyelitis (ME)); mood disorders such as depression; anxiety disorders; panic; psychoses such as Schizophrenia; as well as circadian rhythm related sleep disorders, including jetlag related disorders and nightshift associated conditions. In some further embodiments, the substituted compounds may also help reducing the required amount of sleep and mitigate somnolence. In some further embodiments, the substituted compounds may also to useful to improve, reduce, or mitigate other physiological effects, side effects, or symptoms of a sleeping disorder, such as aching muscles; confusion; memory lapses or losses; depression; development of false memory; hypnagogic and hypnopompic hallucinations during falling asleep and waking; hand tremor; headaches; malaise; stye; periorbital puffiness; increased blood pressure; increased stress hormone levels; increased risk of diabetes; lowering of immunity; increased susceptibility to illness; increased risk of fibromyalgia; irritability; rapid involuntary rhythmic eye movement; obesity; seizures; temper tantrums in children; and symptoms similar to attention-deficit hyperactivity disorder and psychosis.

Polyunsaturated lipids such as polyunsaturated fats, unlike monounsaturated or saturated fats, contain one or more bis-allylic positions—that is —CH₂ groups within the long carbon chain of the fatty acid that are non-conjugated moieties between two unsaturated double bonds. These positions characterize PUFAs and are particularly susceptible to oxidation stress by hydrogen-abstraction to form a free radical. The radical, once formed, is much more reactive than the PUFA itself, and immediately reacts further, usually with oxygen, to form peroxyl radicals, and these are even better than the original disease trigger at propagating more hydrogen-extraction from PUFAs (see Scheme 1).

The chain reaction of PUFA autoxidation is illustrated in Scheme 1 as linoleic acid chain reaction. More PUFAs are entering the cycle at the propagation step. Abstraction of a bis-allylic hydrogen is the rate-limiting step. Peroxides further degrade to cytotoxic aldehydes that further damage proteins and DNA, creating more peroxidation stress.

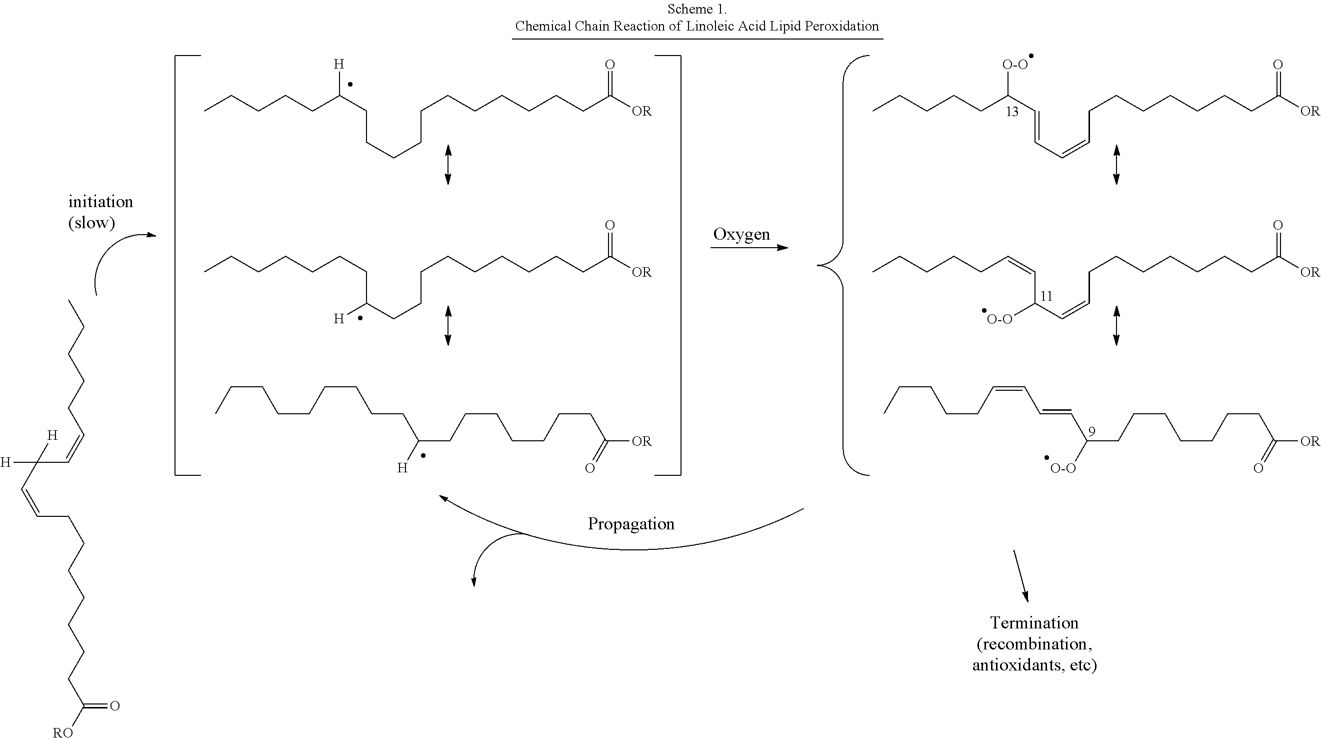

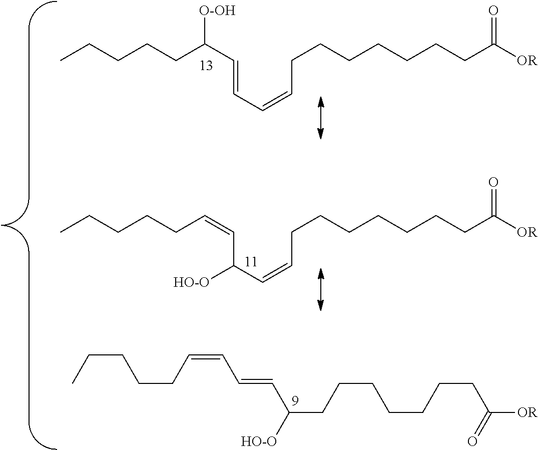

Instead of classical mechanisms of aberrant proteins, differing expression levels, or chemical toxicities can lead directly to disease damage to cells. The factors such as differing expression levels, chemical toxicities, and/or lipid peroxidation may be triggers that do not necessarily lead to clinical disease, unless and until amplified by a free radical mechanism of chemical oxidation of susceptible fatty acids. Since free radical lipid oxidation mechanisms are well understood, and known to involve a cycle of accelerating autocatalytic damage, this common mechanism, independent of the triggers used to initiate it, may be responsible for the massive amount of cellular damage across many indications.

Down-regulating the free radical initiation of lipid peroxidation (Scheme 1) can both prevent and treat cellular damage responsible for disease, and even reverse in vivo phenotypes. Hence stabilizing lipids against such damage becomes a novel treatment modality.

The mechanism of disease onset and progression indicates that lipid peroxidation pathways are linked to disease phenotypes. The pathway includes: 1) the high concentration of ROS generated by cellular energy generation; 2) the concentrated accumulation of highly susceptible polyunsaturated fats in the lipid membranes; and 3) the inadequate protection by antioxidants due to various reasons including the hydrophobic nature of membranes, which limits antioxidant solubility and diffusion into the susceptible domains.

The end metabolic products of damaged polyunsaturated fatty acids, molecules like 4-hydroxy-2-nonenal (4-HNE), 4-hydroxy hexenal (HHE), malondialdeyde (MDA), and many other reactive carbonyl compounds, are candidate markers of neurodegeneration and mitochondrial loss of function, and are observed in virtually all diseases in which lipid peroxidation is implicated.

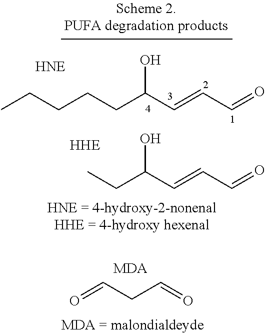

There is a broad body of literature supporting the role of lipid peroxidation in neurodegeneration with brain iron accumulation (NBIA) and related neurodegenerative disease. See Reed et al., “Lipid peroxidation and neurodegenerative disease,” Free Radical Biology & Medicine 51 (2011): 1302-1319. Recent literature also describes the role of lipid peroxidation in INAD, PKAN, and PLA2G6 disorders. See Kinghom et al., “Loss of PLA2G6 leads to elevated mitochondrial lipid peroxidation and mitochondrial dysfunction,” Brain 2015; 138:1801-1816; Kinghom et al., “Mitochondrial dysfunction and defects in lipid homeostasis as therapeutic targets in neurodegeneration with brain iron accumulation,” Rare Diseases 2016, VOL, 4, NO. 1, el 128616. PLA2G6 has been implicated specifically in diseases with brain iron accumulation, such as Friedreich's ataxia, NBIA, and Alzheimer's disease—to name a few—are even more susceptible as iron is a Fenton reaction catalyst for the initiating event of membrane lipid peroxidation pathway.

It is evident that lipid peroxidation plays a significant role in LSD and/or NCL and related neurodegenerative diseases. Malfunction in normal or oxidized lipid processing provokes LPO and exacerbates the toxicity of LPO products, imposing a systemic toxic effect on any lipid membrane containing structure, but particularly on PUFA rich membranes. Examples of LSD diseases include, but are not limited to, Sphingolipidoses, Ceramidase, Farber disease, Krabbe disease (Infantile onset and Late onset), Galactosialidosis, Gangliosides: gangliosidoses, Alpha-galactosidase (including Fabry disease (alpha-galactosidase A), Schindler disease (alpha-galactosidase B)), Beta-galactosidase/GM1 gangliosidosis (Infantile, Juvenile, and Adult/chronic), GM2 gangliosidosis (AB variant, Activator deficiency, Sandhoff disease (Infantile. Juvenile, and Adult/chronic), Tay-Sachs (Juvenile hexosaminidase A deficiency, Chronic hexosaminidase A deficiency), Glucocerebroside (Gaucher disease, Type I, Type II, Type III), Sphingomyelinase (Lysosomal acid lipase deficiency. Early onset or Late onset), Niemann-Pick disease (Type A or Type B), Sulfatidosis, Metachromatic leukodystrophy, Saposin B deficiency, Multiple sulfatase deficiency, Mucopolysaccharidoses (Type I: MPS I Hurler syndrome, MPS I S Scheie syndrome, MPS I H-S Hurler Scheie syndrome; Type II (Hunter syndrome); Type III (Sanfilippo syndrome) MPS III A (Type A), MPS III B (Type B), MPS III C (Type C), MPS ill D (Type D): Type IV (Morquio): MPS IVA (Type A), MPS IVB (Type B); Type VI (Maroteaux-Lamy syndrome); Type VII (Sly syndrome); Type IX (hyaluronidase deficiency)), Mucolipidosis (Type I (sialidosis). Type II (I-cell disease), Type III (pseudo-Hurler polydystrophy/phosphotransferase deficiency), Type IV (mucolipidin 1 deficiency)), Lipidoses (Niemann-Pick disease, type C or Type D), Wolman disease, Oligosaccharide (Alpha-mannosidosis, Beta-mannosidosis, Aspartylglucosaminuria, Fucosidosis), Lysosomal transport diseases (Cystinosis, Pycnodysostosis, Salla disease/sialic acid storage disease, Infantile free sialic acid storage disease). Glycogen storage diseases (Type II Pompe disease, Type IIb Danon disease), Cholesteryl ester storage disease, and lysosomal disease. Examples of NCL type diseases include but are not limited to Type 1 Santavuori-Haltia disease/infantile NCL (CLN1 PPT1), Type 2 Jansky-Bielschowsky disease/late infantile NCL (CLN2/L1NCL TPP1), Type 3 Batten-Spielmeyer-Vogt disease/juvenile NCL (CLN3), Type 4 Kufs disease/adult NCL (CLN4), Type 5 Finnish Variant/late infantile (CLN5), Type 6 Late infantile variant (CLN6), Type 7 CLN7, Type 8 Northern epilepsy (CLN8), Type 8 Turkish late infantile (CLN8), Type 9 German/Serbian late infantile (unknown), Type 10 Congenital cathepsin D deficiency (CTSD), and Batten disease.

The lipid peroxidation chain reaction is the target of the substituted compounds described herein. This chain reaction results in cell damage, death and disease. To halt this damage process substituted compounds as described herein can target the root cause of disease, the amplification of the original disease trigger by lipid peroxidation. Since PUFAs also turn over in diseased as well as normal cells, substituted compounds as described herein can both maintain and restore health and function to them.

The initiation event of the lipid peroxidation chain reaction is caused by ROS abstracting a hydrogen off a bis-allylic (between the double bonds) methylene carbon in the lipid—this is the rate determining step of the chain reaction in lipid peroxidation. If one could slow down the initiation rate, it would have a large effect down-regulating PUFA oxidation by eliminating all of the downstream multiplying ‘cycles’ of damage from each abstraction.

The initial abstraction rate can be reduced by replacing hydrogen atoms at bis-allylic methylene sites with deuterium atoms. Deuterium is naturally present and is recognized by living systems as a normal variation of hydrogen (typically hydrogen in all natural substances consists of 1 deuterium per 7000 hydrogens). Deuterium is also responsible for a well-known “isotope effect” (IE): reactions involving cleavage of a C—H bond are slowed down substantially when H is replaced with D. This substitution reduces the ability of the C—H bond to be broken.

In some embodiments, substituted compounds as described herein (e.g., PUFAs) that are specifically substituted with deuterium at bis-allylic positions can be made in large quantities using well-optimized drug synthetic methods. This modification is both “natural” (deuterium exists in nature) and “game-changing”: whereas the lipid peroxidation process is autocatalytic, the stabilization of the initiating step is ‘anti-’ catalytic, causing at each step a multiplicative >10-fold isotope reduction, essentially shutting down the chain process quickly. Hence, the susceptible target bonds of the chain reaction are “fire-proofed” against the damage of ROS. Importantly, enzymatic processes involving PUFAs, such as β-oxidation, transformations involving other enzymes (all stoichiometric 1:1 enzyme:substrate reactions) are largely unaffected, in addition, this ‘fireproofing’ process requires only a fraction of the total PUFAs be deuterated for the chain reaction of lipid peroxidation to be effectively shut down.

Substituted compounds as described herein that are deuterated (e.g., deuterated PUFAs) thus represent a novel type of sensitive and specific drug which is structurally similar to corresponding compounds having only a natural level of deuteration, but they prevent damaging, non-enzymatic oxidation processes without interfering substantially with biologically necessary enzymatic transformations. For example, because PUFAs in membranes turn over rapidly-even when the cells do not—deuterated PUFAs rapidly replace the original hydrogen containing molecules in all compartments in all tissues. All of the active transport used to transfer normal PUFAs from orally ingested foods work the same on deuterated PUFAs, and transport them wherever they are needed. As a result, D-PUFAs rapidly incorporate into brain, retina, and other difficult to treat tissues.

The substituted compounds as described herein (e.g., deuterated PUFAs, 11,11-D2-linoleic acid ethyl ester) is unique in drug discovery and treatment. Some PUFAs, such as linoleic acid, are part of the human diet that have no pharmacological effect, yet in the deuterated form they may act as sensitive and specific drugs. These type of substituted compounds also do not have any observable side effects.

Linoleic acid (LA) is essential for human diets and is designated as GRAS without a known toxic upper limit for nutritional use. LA was identified in the 1920s and there have been more than 1,300 published human studies of LA. There have been more than 23,000 published human studies of omega-3 PUFAs of which about 2,500 were randomized controlled clinical trials comparing omega-3 PUFAs to LA. No LA-related safety issues were identified in these studies.

Substituted compounds as described herein can be effective in treating a disease or condition associated with a lysosomal storage disease or a condition associated with impaired PLA2G6 activity. The effect of the substituted compounds (e.g., deuterated LA and ester thereof) in promoting cell survival, reducing and/or preventing the damage arising from the free radical chain reaction has been demonstrated. Coenzyme Q deficient coq mutant yeast strains are highly sensitive to oxidation damage from exogenous PUFAs because they lack antioxidant control and the critical hydrophobic intracellular mitochondrial membrane domains are not accessible to other hydrophilic antioxidants. In a coq mutant yeast model, cells that are otherwise healthy when grown on MUFAs (mono-unsaturated fatty acids) and SFAs (saturated fatty acids), do not grow well in the presence of a PUFA (e.g. LA). However, a substituted compound (e.g., deuterated LA and ester thereof) is effective in increasing and/or preserving the viability of the cells.

In addition, a substituted compound as described herein can be effective in reducing and preventing oxidative stress and damages associated with iron accumulation. Yeast, murine, and human in vitro models of Friedreich's ataxia (FRDA) demonstrate that a substituted compound as described herein (e.g., D-PUFA and ester thereof) has been effective in managing the oxidative stress associated with increased iron. In FRDA damaging iron accumulations are observed in the brain tissue, similar to that of INAD. In the yeast model of FRDA, treatment with a substituted compound as described herein (e.g., D-PUFA and ester thereof) and D4-ALA (deuterated linolenic acid such as 11,11,14,14-D4-linolenic acid) can result in decreased lipid peroxidation. In the murine FRDA cell model, a substituted compound as described herein (e.g., 11,11-D2-linoleic acid, 11,11,14,14-D4-linolenic acid, or ester thereof) can protect cells from loss of viability. In the human fibroblast FRDA model, a substituted compound as described herein (e.g., 11,11-D2-linoleic acid, 11,11,14,14-D4-linolenic acid, or ester thereof) can rescue cells from loss of viability. See Cotticelli et al., Redox Biology. 2013 (1):398-404. In another murine model of FRDA, treatment, with D4-ALA can prevent lipid peroxidation. See Abeti et al., Cell Death Dis. 2016 May 26; 7:e2237.

The effect of substituted compounds as described herein (e.g., D-PUFA and ester thereof) on mitochondrial function under oxidative stress conditions was reported by Andreyev. See Andreyev et al., Free Radio Biol Med. 2015 Jan. 8. S0891-5849(15)00003-9. Levels of F2-IsoProstane oxidation products can be decreased dramatically in the deuterated-treated cells, indicating that treatment with the substituted compounds as described herein can decrease the free radical chain processes in a cell as a whole. In H9C2 myoblasts, treatment with t-ButOOH can cause both respiratory inhibition and increased membrane leak. Substituted compounds as described herein (e.g., D-PUFA and ester thereof) can protect mitochondrial function from stress caused by t-ButOOH; maximal respiration is preserved and/or increase in membrane leak is diminished. Other oxidative stress paradigms can also be operative and D4-ALA and ester thereof can be protective against oxidative stress, confirming that the combination of non-deuterated and deuterated PUFAs can be effective in protecting against oxidative stress. A small amount of D-PUFA can provide significant protection against a very severe oxidative stress induced by Fe²⁺ in the presence of an unprotected polyunsaturated fatty acid.

An in vivo model of mitochondrial dysfunction shows that substituted compounds as described herein (e.g., D-PUFA and ester thereof) reduce oxidative stress-related injury. In a model of Parkinson's disease, C57BL/6 mice are treated with the neurotoxin 1-methyl-4-phenyl-1,2,3,6-tetrahydropyridine (MPTP). See Shchepinov et al., Toxicol Lett. 2011 Nov. 30; 207(2):97-103. MPTP's active metabolite, MPP⁺, inhibits complex I of the mitochondrial electron transport chain. Measurement of brain deuterium concentration after administration of substituted compounds as described herein (e.g., D-PUFA and ester thereof) in the diet for 12-13 days indicates that deuterium is incorporated. The animals fed with substituted compounds as described herein (e.g., D-PUFA and ester thereof) show less MPTP effect when compared with the controls. Treatment with substituted compounds as described herein (e.g., D-PUFA and ester thereof) may also rescue the dopaminergic phenotype.

Single- and repeat-dose studies are conducted in mice and rats using both oral gavage and dietary administration of substituted compounds as described herein (e.g., D-PUFA and ester thereof) for up to 26 weeks. The substituted compounds studied (e.g., 11,11-D2-linoleic acid) were well tolerated in all of the studies conducted. The NOAELs established in the 8- and 26-week studies correspond to an average consumption of the substituted compounds studied in the amount of ˜362 and ˜452 mg/kg, respectively. The high dose diets in these studies contained no natural LA. There were no signs observed in this study of essential fatty acid (linoleic acid) deficiency, which is characterized by changes, notably to the skin including alopecia and scaly tails, which appear within months of sustained feeding of a diet lacking LA. This is consistent with the substituted compounds as described herein (e.g., D-PUFA and ester thereof) (di-deutero linoleic acid) being, for dietary purposes, equivalent to and biologically interchangeable with normal dietary LA as a sole source of LA by the rats in this study. Analysis of tissue uptake and distribution indicates that administration of substituted compounds as described herein (e.g., D-PUFA and ester thereof) do not appear to alter the enzymatic processing or availability of PUFAs as measured in the heart, brain, lung, kidney, and liver. Substituted compounds as described herein (e.g., D-PUFA and ester thereof) and their derivatives are incorporated into tissues and do not result in any significant morphological or functional changes in the treated animals versus their controls. The data show that the enzymatic processing of substituted compounds as described herein (e.g., D-PUFA and ester thereof), and the subsequent selective PUFA species incorporation patterns for each of the tissues tested, are the same for the low dose, high dose and control groups for both 8-week and 26-week studies. The body weights, organ weights, percent of each organ composed of PUFAs, distribution of PUFA species in each tissue, and the PUFA composition of red blood cells are not changed versus controls.

These data indicate that substituted compounds as described herein (e.g., D-PUFA and ester thereof) can be effective in inhibiting the free radical degradation of lipids but do not impact the metabolic enzymatic processing of lipids. Thus, substituted compounds as described herein (e.g., D-PUFA and ester thereof) appear to be processed identically to dietary-sourced essential fatty add LA, and hence share the well-known characteristics and safety profile of LA.

Substituted compounds as described herein (e.g., D-PUFA and esters thereof) have been demonstrated in many neurodegenerative disease preclinical models to mitigate both cell death and disease symptoms. The gene defect underlying >90% of INAD disease, PLA2G6, causes increased cell death due from inability to mop up lipid peroxidation. These effects can be reversed over controls in INAD stem cell and fibroblast studies with D-PUFA dosing, and climbing performance in a drosophila model improved. Since D-PUFAs have been proven safe in preclinical toxicity studies and a clinical phase 1/2 study in Friedreich's ataxia, and showed benefit in multiple INAD models, the treatment of a disease or condition associated with an impaired Phospholipase A2 Group VI (PLA2G6) activity such as INAD and PLAN, or a disease or condition associated with an-lysosomal storage disease and/or NCL disease can also be effective.

### Definition

The section headings used herein are for organizational purposes only and are not to be construed as limiting the subject matter described.

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least,” For example, when used in the context of a process, the term “comprising” means that the process includes at least the recited steps, but may include additional steps. When used in the context of a compound, composition, formulation, or device, the term “comprising” means that the compound, composition, formulation, or device includes at least the recited features or components, but may also include additional features or components.

As used herein, common abbreviations are defined as follows:
ANOVA analyst s of variance
BID Twice daily
INAD Infantile neuroaxonal dystrophy
LOTS Late onset Tay-Sachs disease
LPO Lipid Peroxidation
LSD Lysosomal storage disease or disorder
RBC Red blood cell
PLA2G6 Phospholipase A2 Group VI
PLAN PLA2G6 associated neurodegeneration
PK Pharmacokinetics
PUFA Polyunsaturated Fatty Acid
T25FW Timed 25-foot walk

The term “about” as used herein, refers to a quantity, value, number, percentage, amount, or weight that varies from the reference quantity, value, number, percentage, amount, or weight by a variance considered acceptable by one of ordinary skill in the art for that type of quantity, value, number, percentage, amount, or weight. In various embodiments, the term “about” refers to a variance of 20%, 15%, 10%, 9%, 8%, 7%, 6%, 5%, 4%, 3%, 2% or 1% relative to the reference quantity, value, number, percentage, amount, or weight.

The term “oral dosage form,” as used herein, has its ordinary meaning as understood by those skilled in the art and thus includes, by way of non-limiting example, a formulation of a drug or drugs in a form administrable to a human, including pills, tablets, cores, capsules, caplets, loose powder, solutions, and suspensions.

As used herein, the term “ester” refers to the structure —C(═O)OR, wherein R may include unsubstituted or substituted C₁₋₃₀ alkyl (branched or straight), unsubstituted or substituted substituted C₆₋₁₀ aryl, unsubstituted or substituted 5 to 10 membered heteroaryl, unsubstituted or substituted C₃₋₁₀ carbocyclyl, or unsubstituted or substituted 3 to 10 membered heterocyclyl.

As used herein, the term “thioester” refers to the structure —C(═O)SR, wherein R may include unsubstituted or substituted C₁₋₃₀ alkyl (branched or straight), unsubstituted or substituted substituted C₆₋₁₀ aryl, unsubstituted or substituted 5 to 10 membered heteroaryl, unsubstituted or substituted C₃₋₁₀ carbocyclyl, or unsubstituted or substituted 3 to 10 membered heterocyclyl.

As used herein, the term “amide” refers to the structure —C(O)NR¹R² or —S(O)NR¹R², and R¹ and R² can independently be unsubstituted or substituted C₁₋₃₀ alkyl (branched or straight), unsubstituted or substituted substituted C₆₋₁₀ aryl, unsubstituted or substituted 5 to 10 membered heteroaryl, unsubstituted or substituted C₃₋₅₀ carbocyclyl, or unsubstituted or substituted 3 to 10 membered heterocyclyl.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

The term “pediatric patient” as used herein means a human patient that is 17 years old or younger. In certain non-limiting embodiments, the patient is 16 years old or younger, or 15 years old or younger, or 14 years old or younger, or 13 years old or younger, or 12 years old or younger, or 11 years old or younger, or 10 years old or younger, or 9 years old or younger, or 8 years old or younger, or 7 years old or younger, or 6 years old or younger, or 5 years old or younger, or 4 years old or younger, or 3 years old or younger, or 2 years old or younger, or 1 year old or younger, or 6 months old or younger, or 4 months old or younger, or 2 months old or younger, or 1 months old or younger. In particular embodiments, the pediatric patient is between about 12 to about 17 years of age. In one embodiment, the pediatric patient has an age selected from the group consisting of between about 12 to about 17 years of age and about 2 years of age or younger.

As used herein, the act of “providing” includes supplying, acquiring, or administering (including self-administering) a composition described herein.

As used herein, the term “administering” a drag includes an individual obtaining and taking a drug on their own. For example, in some embodiments, an individual obtains a drag from a pharmacy and self-administers the drug in accordance with the methods provided herein.

The term “therapeutically effective amount” as used herein, refers to an amount of a substituted compound described herein sufficient to treat, ameliorate a disease or condition described herein, or to exhibit a detectable therapeutic effect. The effect may be detected by any means known in the art. In some embodiments, the precise effective amount for a subject can depend upon the subject's body weight, size, and health; the nature and extent of the condition; and the therapeutic or combination of therapeutics selected for administration. Therapeutically effective amounts for a given situation may be determined by routine experimentation that is within the skill and judgment of the clinician. In some embodiments, the substituted compound is a polyunsaturated acid (PUFA) or an ester, thioester, amide, or other prodrug thereof, or combinations thereof for treating, or ameliorating the diseases or conditions described herein. In some further embodiment, the substituted compound is 11,11-D2-linoleic acid or an ester thereof.

“Treat,” “treatment,” or “treating,” as used herein refers to administering a compound or pharmaceutical composition to a subject for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject already suffering from a disease or condition.

The pharmaceutical composition described herein are preferably provided in unit dosage form. As used herein, a “unit dosage form” is a composition/formulation containing an amount of a compound that is suitable for administration to an animal, preferably mammal subject, in a single administration, according to good medical practice. The preparation of a single or unit dosage form however, does not imply that the dosage form is administered once per day or once per course of therapy, or that the unit dosage form contains ail of the dose to be administered at a single time. Such dosage forms are contemplated to be administered once, twice, thrice or more per day, and may be given more than once during a course of therapy, though a single administration is not specifically excluded. In addition, multiple unit dosage forms may be administered at substantially the same time to achieve the full dose intended (e.g., two or more tablets may be swallowed by the patient to achieve a complete dose). The skilled artisan will recognize that the formulation does not specifically contemplate the entire course of therapy and such decisions are left for those skilled in the art of treatment rather than formulation.

In any of the embodiments described herein, methods of treatment can alternatively entail use claims, such as Swiss-type use claims. For example, a method of treating a subject having an impaired PLA2G6 activity can alternatively entail the use of a compound in the manufacture of a medicament for the treatment of the disease(s) or conditions) described herein, or a compound for use in the treatment of the disease(s) or condition(s) described herein.

### Methods of Treatment

Some embodiments relate to a method of treating a subject having, or at risk for, a disease or condition associated with an impaired Phospholipase A2 Group VI activity, comprising: selecting a subject having, or at risk for, a disease or condition associated with an impaired Phospholipase A2 Group VI activity; and administering to the subject an effective amount of a substituted compound selected from the group consisting of a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, and combinations thereof, wherein the substituted compound comprises at least one substituent that reduces oxidation of the substituted compound. In some embodiments, the subject has infantile neuroaxonal dystrophy or PLA2G6 associated neurodegeneration. In one embodiment, the subject has infantile neuroaxonal dystrophy. In some such embodiments, the infantile neuroaxonal dystrophy is caused by PLA2G6 mutation.

Some embodiments relate to a method of treating a subject having, or at risk for, a disease or condition associated with a lysosomal storage disease and/or neuronal ceroid lipofuscinosis disease, comprising: selecting a subject having, or at risk for, a disease or condition associated with a lysosomal storage disease or neuronal ceroid lipofuscinosis; and administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, or combinations thereof, wherein the substituted compound comprises at least one substituent that reduces oxidation of the substituted compound. In some embodiments, the subject has Tay-Sachs, Gaucher disease, Sandhoff disease, or Niemann-Pick disease. In one embodiment, the subject has Tay-Sachs disease, for example, late onset Tay-Sachs disease. In some such embodiments, Tay-Sachs disease is caused by genetic mutation in the HEXA gene. In some embodiments, the LSD is GM1 gangliosidosis. In some embodiments, the LSD is GM2 gangliosidosis. In some embodiments, the LSD is sphingolipidose disease.

Some embodiments relate to a method of treating a subject having, or at risk for, a sleeping disorder, comprising: selecting a subject having, or at risk for, a sleeping disorder; and administering to the subject an effective amount of a substituted compound selected from the group consisting of a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, and combinations thereof, wherein the substituted compound comprises at least one substituent that reduces oxidation of the substituted compound. In some embodiments, the subject has acute or chronic dyssomnia. In some embodiments, the subject has obstructive sleep apnoea syndrome.

In some embodiments of the methods described herein, the administering step comprises repeated administration. In some embodiments, the subject has or is at risk for at least one of neuropathy or a neurodegenerative disease and the amount of the substituted compound is effective to prevent, ameliorate or inhibit the progression of neuropathy or the neurodegenerative disease.

In some embodiments of the methods described herein, the substituted compound comprises one or more isotopes, and the amount of the isotope is significantly above the naturally-occurring abundance level of the isotope. For example, in some embodiments, the amount of the isotope is two or more times greater than the naturally-occurring abundance level of the isotope. In some embodiments, the isotope is selected from deuterium, ¹³C, and a combination thereof. In some embodiments, the isotope atom is deuterium. The substituted compound, for example, isotopically modified PUFAs such as deuterated PUFAs may reduce oxidation by at least 5%, 10%, 15%, 20%, 25%, 30%, 35%, 40%, 45%, 50%, 55%, 60%, 65%, 70%, 75%, 80%, 85%, 90% or 95%.

In some embodiments, the method described herein comprises identifying or selecting from treatment a subject having an impaired PLA2G6 activity. In some such embodiments, identifying or selecting the subject having the impaired PLA2G6 activity may include sequencing the subject's DNA or using a genetic test to identify and screen for patients having a mutation of PLA2G6 gene. In some embodiments, identifying a patient having an impaired PLA2G6 activity is established in a proband by identification of biallelic pathogenic variants in PLA2G6 on molecular genetic testing. In some embodiments, identifying a patient having an impaired PLA2G6 activity can be established in a proband with no identified PLA2G6 pathogenic variants by electron microscopic examination of nerve biopsies for dystrophic axons (axonal spheroids).

In some embodiments, the method described herein comprises identifying or selecting for treatment a subject having LSD and/or NCL. In some such embodiments, identifying or selecting the subject having LSD and/or NCL may include sequencing the subject's DNA or using a genetic test to identify and screen for patients having a gene mutation associated with a LSD and/or NCL type disease. In some such embodiments, identifying or selecting the subject having LSD and/or NCL comprises sequencing the subject's DNA or using a genetic test to identify and determine the expression or activity of PGRN or detecting one or more mutation in the genomic DNA or gene encoding PGRN. In some embodiments, identifying or selecting the subject having LSD and/or NCL comprises sequencing the subject's DNA or using a genetic test to identify and determine the expression or activity of PGRN or detecting one or more mutation in the genomic DNA or gene encoding HEX (e.g., HEXA, HEXB, or HEXS). In some further embodiments, identifying or selecting the subject having LSD and/or NCL comprises sequencing the subject's DNA and detecting one or more mutation in the genomic DNA or gene encoding HEX (e.g., HEXA, HEXB, or HEXS). In some embodiments, the gene mutation associated with a LSD and/or NCL can be GBA mutation. In some embodiments, the gene mutation associated with a LSD and/or NCL can be PGRN mutation. In some embodiments, identifying the subject having LSD and/or NCL comprises sequencing the subject's DNA or using a genetic test to identify and determine the expression or activity of SMPD1, NPC1, or NPC2 or detecting one or more mutation in the genomic DNA or gene encoding SMPD1, NPC1, or NPC2.

In some embodiments of the methods described herein, the subject has or is at risk for at least one of a neuropathy or a neurodegenerative disease associated with the impaired PLA2G6 activity. In some embodiments, the subject has an infantile neuroaxonal dystrophy (INAD) or PLA2G6 associated neurodegeneration (PLAN). In some embodiments, the neuropathy or a neurodegenerative disease associated with the impaired PLA2G6 activity does not include Alzheimer's disease. In some embodiments, neuropathy or a neurodegenerative disease associated with the impaired PLA2G6 activity does not include Parkinson's disease.

In some embodiments, the amount of the substituted compound administered to the subject is effective to alleviate one or more symptoms of the disease or condition associated with the impaired Phospholipase A2 Group VI (PLA2G6) activity. In some such embodiments, the symptom of the disease or condition is selected from the group consisting of hypotonia, nystagmus, strabismus, psychomotor regression, and low spontaneous motor activity.

In some embodiments, the amount of the substituted compound administered to the subject is effective to alleviate one or more symptoms associated with the LSD and/or NCL. The symptoms for LSD and/or NCL may be different depending on the patient conditions. In some embodiments, the symptom of the disease or condition is at least one selected from the group consisting of difficulties with physical movement (e.g., joint stiffness and pain), seizures, dementia, mental retardation, high fatality, problems vision (e.g., blindness) or hearing (deafness), and problem with bulbar function.

In some embodiments, the amount of the substituted compound administered to the subject is effective to alleviate one or more symptoms or side effects of a sleeping disorder or insufficient sleep. In some such embodiments, the side effects or symptom of a sleeping disorder or insufficient sleep are selected from the group consisting of aching muscles; confusion; memory lapses or losses; depression; development of false memory; hypnagogic and hypnopompic hallucinations during falling asleep and waking; hand tremor; headaches; malaise; stye; periorbital puffiness; increased blood pressure; increased stress hormone levels; increased risk of diabetes; lowering of immunity; increased susceptibility to illness; increased risk of fibromyalgia; irritability; rapid involuntary rhythmic eye movement; obesity; seizures; temper tantrums in children; and symptoms similar to attention-deficit hyperactivity disorder and psychosis.

In some further embodiments, the amount of the substituted compound administered to the subject is effective to increase the muscle functions of the subject. In some embodiments, the muscle function is selected from the group consisting of eye tracking, control, lifting, fine motor skill, and muscle strength.

In some further embodiments, the amount of the substituted compound administered to the subject is effective to increase the neural function of the subject. In some embodiments, the neural function is selected from the group consisting of responsiveness to verbal commands, bulbar function, and verbal cognition. In some embodiments, the neural function is the bulbar function.

In some embodiments, administration of the substituted compounds as described herein can be used in combination with one or more additional therapies for treating INAD selected from a pharmacologic treatment of spasticity and seizures; a trial of oral or intrathecal baclofen for dystonia associated with atypical INAD; a treatment by a psychiatrist for those with later-onset neuropsychiatric symptoms; a fiber supplement and/or stool softener treatment for constipation; control of secretions with transdermal scopolamine patch as needed; feeding modifications as needed to prevent aspiration pneumonia and achieve adequate nutrition, and a combination thereof. In some embodiments, administration of a substituted compound as described herein can be used in combination with one or more additional therapies for treating PLAN selected from the group consisting of treatment with dopaminergic agents; treatment of neuropsychiatric symptoms by a psychiatrist; evaluation by physical therapy for management of postural instability and gait difficulties; occupational therapy to assist with activities of daily living; feeding modifications as needed to prevent aspiration pneumonia and achieve adequate nutrition, and a combination thereof.

In some embodiments, administration of the substituted compounds as described herein can be used in combination with one or more additional therapies for treating LSD and/or NCL diseases. For example, a subject suffering from a sleeping disorder described herein may be administered with antioxidants, melatonin, glycine, sleep medication, antidepressant to improve or regulate sleep-wake cycle and/or mitigate the side effects associated with a sleeping disorder or insufficient sleep.

Doses

In some embodiments of the methods described herein, the therapeutically effective amount of a substituted compound administered to the subject is about 0.1 g, 0.2 g, 0.5 g, 1.0 g, 1.5 g, 2.0 g, 2.5 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g 18 g, 18.5 g, 19 g, 19.5 g, or 20 g, or a range defined by any of the two preceding values. In some embodiments, the amount of the substituted compound administered is from about 0.1 g to about 20 g, from about 1 g to about 10 g, from 2 g to about 5 g. In some further embodiments, the amount of the substituted compound administered is from about 1.8 g to about 4.5 g. In some embodiments, the substituted compound is in a single unit dosage form. In some other embodiments, the substituted compound is in two or more unit dosage forms (i.e., a divided dose). For example, where a dose is about 5 g, it may be provided in the form of four or five tablets, each containing about 1.25 g or 1 g of the substituted compound. In some such embodiments, a dose of 1 g to 10 g comprises administering 1, 2, 3, 4 or 5 unit dosage forms each comprising from about 1 g to about 2 g of the substituted compound, or about 2, 3, or 4 unit dosage forms each comprising from about 0.5 g to about 2.5 g of the substituted compound. In another example, a dose of 2 g to 5 g comprises administering 1, 2, 3, 4 or 5 unit dosage forms each comprising from about 1 g to about 2 g of the substituted compound. In some embodiments, the unit dosage form is a tablet, a capsule, a pill, or pellets. In some further embodiment, the unit dosage form for oral administration, i.e., oral dosage form.

In some embodiments of the methods described herein, the substituted compound may be administered once per day. In some other embodiments, the substituted compound may be administered two or more times per day, for example, twice a day or three times a day. In some embodiments, the therapeutically effective amount of the substituted compound administered per day is about 10 g, 2.0 g, 3.0 g, 3.5 g, 4.0 g, 4.5 g, 5.0 g, 5.5 g, 6.0 g, 6.5 g, 7.0 g, 7.5 g, 8.0 g, 8.5 g, 9.0 g, 9.5 g, 10 g, 10.5 g, 11 g, 11.5 g, 12 g, 12.5 g, 13 g, 13.5 g, 14 g, 14.5 g, 15 g, 15.5 g, 16 g, 16.5 g, 17 g, 17.5 g 18 g, 18.5 g, 19 g, 19.5 g, 20 g, 25 g, 30 g, 35 g, 40 g, 45 g, or 50 g, or a range defined by any of the two preceding values. In some such embodiments, the amount of the substituted compound administered per day is from about 1 g to about 20 g, from about 2 g to about 10 g, from about 3 g to about 8 g, from about 4 g to about 7 g, or from about 5 g to about 6 g. In one embodiment, the amount of 11,11-D2-linoleic acid or the ester thereof administered per day is from about 2 g to about 10 g. In another embodiment, the amount of 11,11-D2-linoleic acid or the ester thereof administered per day is from about 1.8 g to about 9 g.

In some embodiments of the method described herein, the substituted compound may be administered for at least 1 week, 2 weeks, 3 weeks, 4 weeks, 5 weeks, 6 weeks, 7 weeks or 8 weeks. In some embodiments, the method further comprises detecting the steady state plasma level of the substituted compound, or the level of the substituted compounds within red blood cell membrane to determine the incorporation level of the substituted compound. In some such embodiments, the plasma level of the substituted compound reaches a steady state after 1, 2, 3 or 4 weeks. In one embodiment, the plasma level of the substituted compound may reach a steady state within 15 days, 20 days, 30 days, 40 days, 50 days or 60 days.

In some embodiments, the dosage of the substituted compound is in the range from about 10 mg/kg to about 200 mg/kg, or from about 20 mg/kg to about 100 mg/kg. In some embodiments, the dosage of the substituted compound is in the range from about 30 mg/kg to about 80 mg/kg. In some embodiments, the daily dose of the substituted compound is in the range of about 1 g to about 10 g. In some embodiments, the daily dose of the substituted compound is about 1.8 g or about 9 g. In some embodiments, the daily dose of the substituted compound is about 1.8 g. In one embodiment, the daily dose of the substituted compound is about 4.5 g administered twice a day. In another embodiment, the daily dose of the substituted compound is about 2.7 g administered twice a day.

In some embodiments, the substituted compound is co-administered to the subject with at least one antioxidant. In some embodiments, the antioxidant is selected from Coenzyme Q, idebenone, mitoquinone, mitoquinol, plastoquinone, resveratrol, vitamin E, and vitamin C, and combinations thereof. In some such embodiments, the antioxidant may be taken concurrently, prior to, or subsequent to the administration of the substituted compound. In some embodiments, the antioxidant and the substituted compound may be in a single dosage form. In some embodiments, the single dosage form is selected from the group consisting of a pill, a tablet, and a capsule.

### Substituted Compounds

In some embodiments, the substituted compound comprises at least one isotope, and the amount of the isotope is significantly above the naturally-occurring abundance level of the isotope. For example, in some embodiments, the amount of the isotope is two or more times greater than the naturally-occurring abundance level of the isotope. In some such embodiments, the substituted compound comprises an amount of deuterium that is significantly above the naturally-occurring abundance level of the deuterium. For example, in some embodiments, the amount of the deuterium in the substituted compound is two or more times greater than the naturally-occurring abundance level of the deuterium.

In some embodiments, the substituted compound is an isotopically modified polyunsaturated fatty acid ester. In some embodiments, the substituted compound is an isotopically modified polyunsaturated fatty acid. In some embodiments, the polyunsaturated fatty acid ester is a triglyceride, a diglyceride, a monoglyceride or an alkyl ester. In some embodiments, the polyunsaturated fatty acid ester is an ethyl ester.

The term “substituted compound” as used herein, refers to a compound that is modified by substitution at one or more positions to reduce the rate at which the compound is oxidized. The modification can be an isotopic substitution or a non-isotopic chemical modification. Isotopic substitution can refer to one or more substitutions with an isotope such as deuterium or ¹³C. Non-isotopic modification can refer to substitution at an allylic hydrogen with another chemical group or changing the position of an unsaturated bond to eliminate an allylic hydrogen position to reduce oxidation of the substituted compound.

The term “polyunsaturated lipid” as used herein, refers to a lipid that contains one or more unsaturated bonds, such as a double or a triple bond, in its hydrophobic tail. The polyunsaturated lipid may be a polyunsaturated fatty acid (PUFA) or ester thereof.

The term “bis-allylic position” as used herein, refers to the position of the polyunsaturated lipid, such as polyunsaturated fatty acid or ester thereof that corresponds to the methylene groups of 1,4-diene systems. Examples of polyunsaturated lipids having deuterium at one or more bis-allylic positions include but are not limited to 11,11-Dideutero-cis,cis-9,12-Octadecadienoic acid (11,11-Dideutero-(9Z,12Z)-9,12-Octadecadienoic acid; D2-LA); and 11,11,14,14-Tetradeutero-cis,cis,cis-9,12,15-Octadecatrienoic acid (11,11,14,14-Tetradeutero-(9Z,12Z,15Z)-9,12,15-Octadecatrienoic acid; D4-ALA). [§ 94] The term “pro-bis-allylic position” as used herein, refers to the methylene group in a compound that becomes the bis-allylic position upon desaturation. For example, some sites which are not bis-allylic in precursor PUFAs become bis-allylic upon biochemical transformation. The pro-bis-allylic positions, in addition to being deuterated, can be further substituted by carbon-13, each at levels of isotopic abundance above the naturally-occurring abundance level. For example, the pro-bis-allylic positions, in addition to existing bis-allylic positions, can be reinforced by isotopic substitution as shown below in Formula (1), wherein R¹ is —OH, —O-alkyl, -amine, —S-alkyl, or —O-cation (e.g., cation being Na⁺ or K⁺); m is 0 to 10; n is 1 to 5; and p is 0 to 10. In Formula (1), the position of the X atom represents the pro-bis-allylic position, while the position of the Y atom represents the bis-allylic position, each of X¹, X², Y¹, and Y² atoms may independently be hydrogen or deuterium atoms, and at least one of X¹, X², Y¹, or Y² atoms is deuterium. Each Y¹ and Y² for each n unit can independently be hydrogen or deuterium atoms, and each X¹ and X² for each m unit can independently be hydrogen or deuterium atoms.

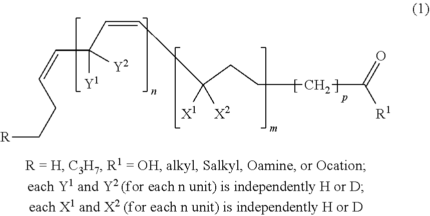

Another example of a substituted compound having bis-allylic and pro-bis-allylic positions is shown in Formula (2), wherein any of the pairs of Y¹-Yⁿ and/or X¹-X^{m} independently represent the bis-allylic and pro-bis-allylic positions of PUFAs respectively and these positions may contain deuterium atoms. X¹, X², . . . Xⁿ, Y¹, Y², . . . Yⁿ atoms can independently be hydrogen or deuterium atoms, and at least one or more of X¹, X², . . . Xⁿ, Y¹, Y², . . . Yⁿ atoms is deuterium. In some embodiments, at least one of Y¹, Y², . . . Yⁿ atoms is deuterium. In some embodiments, p is 0, 1 or 2. In some embodiments, m is 0, 1, 2, or 3. In some embodiments, n is 1, 2, 3, or 4. In some embodiments, n is greater than 1. In some embodiments, n is less than 4.

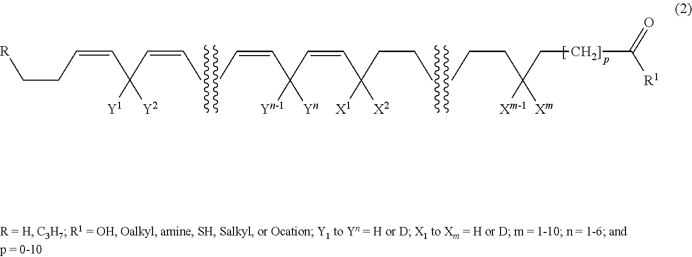

A substituted compound as described herein can be a polyunsaturated lipid that has at least one substitution that reduces oxidation of the substituted compound. In some embodiments, the substituted compound is isotopically modified to reduce oxidation. In some embodiments, the substituted compound is non-isotopically modified at one or more positions to reduce oxidation. The substituted compound, for example, isotopically modified PUFAs such as deuterated PUFAs may reduce oxidation by at least 5%, 10%, 15%, 20%, 25%, 30%, 35%, 40%, 45%, 50%, 55%, 60%, 65%, 70%, 75%, 80%, 85%, 90% or 95%.

In some embodiments, the substituted compound as described herein comprises an isotopically modified polyunsaturated fatty acid, isotopically modified polyunsaturated fatty acid ester, isotopically modified polyunsaturated fatty acid thioester, isotopically modified polyunsaturated fatty acid amide, isotopically modified polyunsaturated fatty acid mimetic, or isotopically modified polyunsaturated fatty acid pro-drug. In some embodiments, the substituted compound as described herein can be an isotopically modified polyunsaturated fatty acid or fatty acid ester. In some embodiments, the substituted compound can be an isotopically modified naturally occurring PUFA. In some embodiments, the substituted compound can have conjugated double bonds. In some embodiments, the substituted compound is an isotopically modified polyunsaturated fatty acid. In some embodiments, the substituted compound is an isotopically modified polyunsaturated fatty acid thioester. In some embodiments, the substituted compound is an isotopically modified polyunsaturated fatty acid amide. In some embodiments, the substituted compound is an isotopically modified polyunsaturated fatty acid mimetic. In some embodiments, the substituted compound is an isotopically modified polyunsaturated fatty acid prodrug.

In some embodiments, the substituted compound can be a deuterated polyunsaturated lipid. In some embodiments, the substituted compound can be a deuterated polyunsaturated fatty acid, a deuterated polyunsaturated fatty acid ester, a deuterated polyunsaturated fatty acid thioester, a deuterated fatty acid amide, a deuterated polyunsaturated fatty acid mimetic, a deuterated polyunsaturated fatty acid pro-drug, or combinations thereof.

In some embodiments, the substituted compound is deuterated at one or more bis-allylic positions. In some embodiments, the substituted compound is further deuterated at one or more pro-bis-allyl positions.

In some embodiments, the substituted compound is a ω-3 fatty acid, a ω-6 fatty acid, a ω-3 fatty acid ester, a ω-6 fatty acid ester, a ω-3 fatty acid amide, a ω-6 fatty acid amide, a ω-3 fatty acid thioester, or a ω-6 fatty acid thioester, or combinations thereof. In some embodiments, the substituted compound is a ω-3 fatty acid, a ω-3 fatty acid ester, a ω-3 fatty acid amide, a ω-3 fatty acid thioester, a prodrug thereof, or a combination thereof. In some embodiments, the substituted compound is a ω-6 fatty acid, a ω-6 fatty acid ester, a ω-6 fatty acid amide, a ω-6 fatty acid thioester, a prodrug thereof, or combinations thereof. In some embodiments, the substituted compound is a linoleic acid, a linolenic acid, an arachidonic acid, an eicosapentaenoic acid, a docosahexaenoic acid, or an ester, amide, thioester, or prodrug thereof or combinations thereof.

In some embodiments, the subject also ingests at least one of an unsubstituted polyunsaturated fatty acid and an unsubstituted polyunsaturated fatty acid ester.

In some embodiments, the amount of the substituted compound is about 5% or greater than the total amount of the polyunsaturated fatty acids and polyunsaturated fatty acid esters administered or delivered to the subject. In some embodiments, the amount of the substituted compound is about 10% or greater than the total amount of the polyunsaturated fatty acids and polyunsaturated fatty acid esters administered to the patient. In some embodiments, the amount of the substituted compound is about 15% or greater than the total amount of the polyunsaturated fatty acids and polyunsaturated fatty acid esters administered to the subject. In some other embodiments, the amount of the substituted compound is equal to or less than about 1% of the total amount of the polyunsaturated fatty acids and polyunsaturated fatty acid esters administered or delivered to the subject.

In some embodiments, the polyunsaturated fatty acid, polyunsaturated fatty acid ester, polyunsaturated fatty acid thioester, polyunsaturated fatty acid amide, polyunsaturated fatty acid mimetic, or polyunsaturated fatty acid pro-drug can be a naturally occurring PUFA. In some embodiments, the polyunsaturated fatty acid, polyunsaturated fatty acid ester, polyunsaturated fatty acid thioester, polyunsaturated fatty acid amide, polyunsaturated fatty acid mimetic, or polyunsaturated fatty acid pro-drug can have conjugated double bonds.

In some embodiments, the substituted compound is deuterated at one or more positions. In some embodiments, the substituted compound is deuterated at one or more bis-allylic positions. In some embodiments, the polyunsaturated fatty acid, polyunsaturated fatty acid ester, polyunsaturated fatty acid thioester, polyunsaturated fatty acid amide, polyunsaturated fatty acid mimetic, or polyunsaturated fatty acid pro-drug is deuterated at one or more positions. In some embodiments, the polyunsaturated fatty acid, polyunsaturated fatty acid ester, polyunsaturated fatty acid thioester, polyunsaturated fatty acid amide, polyunsaturated fatty acid mimetic, or polyunsaturated fatty acid pro-drug is deuterated at one or more bis-allylic positions.

In some embodiments, the substituted compound is a fatty acid or fatty acid ester. In some such embodiments, the ester may be a triglyceride, a diglyceride, a monoglyceride, or an alkyl ester. In some further embodiments, the polyunsaturated fatty acid ester is a methyl or ethyl ester.

In some embodiments, the deuterated fatty acid or fatty acid ester are co-administered to a patient with non-deuterated fatty acids or fatty acid esters.

In some embodiments, the substituted compound comprises between about 1 wt % to about 100 wt %, about 5 wt % to about 90 wt %, about 10 wt % to about 50 wt %, about 20 wt % to about 40 wt % of the total amount of polyunsaturated fatty acid, polyunsaturated fatty acid ester, polyunsaturated fatty acid thioester, polyunsaturated fatty acid amide, polyunsaturated fatty acid mimetic, and polyunsaturated fatty acid pro-drug administered or delivered to the patient. In some embodiments, the substituted compound comprises between about 10 wt % and about 40 wt % of the total amount of polyunsaturated fatty acid, polyunsaturated fatty acid ester, polyunsaturated fatty acid thioester, polyunsaturated fatty acid amide, polyunsaturated fatty acid mimetic, and polyunsaturated fatty acid pro-drug administered to the patient. In some embodiments, the substituted compound comprises about 1 wt %, 2 wt %, 3 wt %, 4 wt %, 5 wt %, 6 wt %, 7 wt %, 8 wt %, 9 wt %, 10 wt %, 15 wt %, 20 wt % or more of the total amount of polyunsaturated fatty acid, polyunsaturated fatty acid ester, polyunsaturated tatty acid thioester, polyunsaturated fatty acid amide, polyunsaturated fatty acid mimetic, and polyunsaturated fatty acid pro-drug administered or delivered to the patient. In some further embodiments, the substituted compound is a deuterated fatty acid or fatty acid ester.

In some embodiments, the deuterated fatty acid or fatty acid ester comprises between about 1 wt % to about 100 wt %, about 5 wt % to about 90 wt %, about 10 wt % to about 50 wt %, about 20 wt % to about 40 wt % of the total amount of fatty acids or fatty acid esters administered or delivered to the subject. In some embodiments, the deuterated fatty acid or fatty acid ester comprises about 1 wt %, 2 wt %, 3 wt %, 4 wt %, 5 wt %, 6 wt %, 7 wt %, 8 wt %, 9 wt %, 10 wt %, 15 wt %, 20 wt % or more of the total amount of fatty acids or fatty acid esters administered or delivered to the subject.

In some embodiments, a cell or tissue of the patient maintains a sufficient concentration of the deuterated fatty acid or fatty acid ester to prevent or reduce autoxidation of the naturally occurring non-deuterated fatty acid or fatty acid ester.

In some embodiments, the deuterated substituted compound has an isotopic purity in the range of about 20% to about 99%.

In some embodiments, the fatty acid or fatty acid ester is one or more selected from the group consisting of 11,11-D2-linolenic acid, 14,14-D2-linolenic acid, 11,11,14,14-D4-linolenic acid, 11,11-D2-linoleic acid, 14,14-D2-linoleic acid, and 11,11,14,14-D4-linoleic acid. In some embodiments, the substituted compound is an omega-3 fatty acid or an omega-3 fatty acid ester. In some embodiments, the substituted compound is an omega-6 fatty acid or an omega-6 fatty acid ester.

In some embodiments, the substituted compound is a linoleic acid, a linolenic acid, an arachidonic acid (ARA), a docosahexaenoic acid (DHA), or an eicosapentaenoic acid, (EPA), or combinations thereof. In some embodiments, the substituted compound is an arachidonic acid, a docosahexaenoic acid, an eicosapentaenoic acid containing one or more deuterium. In some embodiments, the substituted compound is an arachidonic acid, a docosahexaenoic acid, an eicosapentaenoic acid, each containing one or more deuterium at one or more bis-allylic positions.

In some embodiments, the substituted compound is selected from the group consisting of 11,11-D2-linolenic acid, 14,14-D2-linolenic acid, 11,11,14,14-D4-linolenic acid, 11,11-D2-linoleic acid, an ester thereof, and a combination thereof. In some embodiments, the substituted compound is selected from the group consisting of 7,7-D2-arachidonic acid; 10,10-D2-arachidonic acid; 13,13-D2-arachidonic acid; 7,7,10,10-D4-arachidonic acid; 7,7,13,13-D4-arachidonic acid; 10,10,13,13-D4-arachidonic acid; 7,7,10,10,13,13-D6-arachidonic acid; 7,7,10,10,13,13,16,16-D8-eicosapentaenoic acid; 6,6,9,9,12,12,15,15,18,18-D10-docosahexaenoic acid; an ester thereof, and combinations thereof. In some embodiments, the substituted compound is 11,11-D2-linoleic acid ethyl ester. In some embodiments, the substituted compound is 11,11,14,14-D4-linolenic acid ethyl ester. In some embodiments, the substituted compound is 7,7,10,10,13,13-D6-arachidonic acid; 7,7,10,10,13,13,16,16-D8-eicosapentaenoic acid; 6,6,9,9,12,12,15,15,18,18-D10-docosahexaenoic acid; or ester thereof. In some embodiments, the substituted compound is 7,7,10,10,13,13-D6-arachidonic acid; 7,7,10,10,13,13,16,16-D8-eicosapentaenoic acid or ester thereof. In some embodiments, the substituted compound is 7,7,10,10,13,13,16,16-D8-eicosapentaenoic acid or ester thereof. In some embodiments, the substituted compound is 6,6,9,9,12,12,15,15,18,18-D10-docosahexaenoic acid; or ester thereof.

In some embodiments, the fatty acid or fatty acid ester is an omega-3 fatty acid. In some embodiments, the omega-3 fatty acid is alpha linolenic acid. In some embodiments, the omega-3 fatty acid is ARA. In some embodiments, the omega-3 fatty acid is EPA. In some embodiments, the omega-3 fatty acid is DHA.

In some embodiments, the fatty acid or fatty acid ester is an omega-6 fatty acid. In some embodiments, the omega-6 fatty acid is linoleic acid. In some embodiments, the omega-6 fatty acid is gamma linolenic acid, dihomo gamma linolenic acid, arachidonic acid, or docosatetraenoic acid. In some embodiments, the fatty acid or fatty acid ester is an omega-6 ARA. In some embodiments, the fatty acid or fatty acid ester is an omega-6 DHA. In some embodiments, the fatty acid or fatty acid ester is an omega-6 EPA.

The substituted compound that is isotopically reinforced at oxidation sensitive positions as described by way of the structures above are heavy isotope enriched at said positions as compared to the natural abundance of the appropriate isotope. In some embodiments, the substituted compound has the deuterium atom present at a level greater than its natural abundance level. Deuterium has a natural abundance of about 0.0156%. Thus, a substituted compound having greater than the natural abundance of deuterium has greater than 0.0156% of its hydrogen atoms replaced or “reinforced” with deuterium, such as 0.02%, but preferably about 5%, 10%, 15%, 20%, 25%, 30%, 35%, 40%, 45%, 50%, 65%, 60%, 65%, 70%, 75%, 80%, 85%, 90%, 95%, or 100% of deuterium with respect to one or more hydrogen atoms in each substituted compound molecule. In other embodiments, the percentage of total hydrogen atoms in a substituted compound that is reinforced with deuterium is at least 0.02%, 0.03% (about twice natural abundance), 0.05%, 0.1%, 1%, 2%, 5%, 10%, 15%, 20%, 25%, 30%, 35%, 40%, 45%, 50%, 65%, 60%, 65%, 70%, 75%, 80%, 85%, 90%, 95%, or 100%.

In some aspects, a composition of substituted compound contains both isotopically modified polyunsaturated lipid and isotopically unmodified polyunsaturated lipid. In some embodiments, isotopic purity refers to the percentage of molecules of an isotopically modified polyunsaturated lipid in the composition relative to the total number of molecules of the isotopically modified polyunsaturated lipid plus polyunsaturated lipid with no heavy atoms. For example, the isotopic purity may be about 5%, 10%, 15%, 20%, 25%, 30%, 35%, 40%, 45%, 50%, 65%, 60%, 65%, 70%, 75%, 80%, 85%, 90%, 95%, or 100% of the molecules of isotopically modified polyunsaturated lipid relative to the total number of molecules of both the isotopically modified polyunsaturated lipid plus polyunsaturated lipid with no heavy atoms. In other embodiments, the isotopic purity is at least 5%, 10%, 15%, 20%, 25%, 30%, 35%, 40%, 45%, 50%, 65%, 60%, 65%, 70%, 75%, 80%, 85%, 90%, 95%, or 100%. In some embodiments, isotopic purity of the polyunsaturated lipid may be from about 10%-100%, 10%-95%, 10%-90%, 10%-85%, 10%-80%, 10%-75%, 10%-70%, 10%-65%, 10%-60%, 10%-55%, 10%-50%, 10%-45%, 10%-40%, 10%-35%, 10%-30%, 10%-25%, or 10%-20% of the total number of molecules of the polyunsaturated lipid in the composition. In other embodiments, isotopic purity of the polyunsaturated lipid may be from about 15%-100%, 15%-95%, 15%-90%, 15%-85%, 15%-80%, 15%-75%, 15%-70%, 15%-65%, 15%-60%, 15%-55%, 15%-50%, 15%-45%, 15%-40%, 15%-35%, 15%-30%, 15%-25%, or 15%-20% of the total number of molecules of the polyunsaturated lipid in the composition. In some embodiments, isotopic purity of the polyunsaturated lipid may be from about 20%-100%, 20%-95%, 20%-90%, 20%-85%, 20%-80%, 20%-75%, 20%-70%, 20%-65%, 20%-60%, 20%-55%, 20%-50%, 20%-45%, 20%-40%, 20%-35%, 20%-30%, or 20%-25% of the total number of molecules of the polyunsaturated lipid in the composition. Two molecules of an isotopically modified polyunsaturated lipid out of a total of 100 total molecules of isotopically modified polyunsaturated lipid plus polyunsaturated lipid with no heavy atoms can have 2% isotopic purity, regardless of the number of heavy atoms the two isotopically modified molecules contain.

In some aspects, an isotopically modified PUFA molecule may contain one deuterium atom, such as when one of the two hydrogens in a methylene group is replaced by deuterium, and thus may be referred to as a “D1” PUFA. Similarly, an isotopically modified PUFA molecule may contain two deuterium atoms, such as when the two hydrogens in a methylene group are both replaced by deuterium, and thus may be referred to as a “D2” PUFA. Similarly, an isotopically modified PUFA molecule may contain three deuterium atoms and may be referred to as a “D3” PUFA. Similarly, an isotopically modified PUFA molecule may contain four deuterium atoms and may be referred to as a “D4” PUFA. In some embodiments, an isotopically modified PUFA molecule may contain five deuterium atoms or six deuterium atoms and may be referred to as a “D5” or “D6” PUFA, respectively.

The number of heavy atoms in a molecule, or the isotopic load, may vary. For example, a molecule with a relatively low isotopic load may contain about 1, 2, 3, 4, 5, or 6 deuterium atoms. A molecule with a moderate isotopic load may contain about 10, 11, 12,13, 14, 15, 16, 17, 18, 19, or 20 deuterium atoms. In a molecule with a very high load, every hydrogen may be replaced with a deuterium. Thus, the isotopic load refers to the percentage of heavy atoms for that type of atom in each substituted compound or polyunsaturated lipid molecule. For example, the isotopic load may be about 5%, 10%, 15%, 20%, 25%, 30%, 35%, 40%, 45%, 50%, 65%, 60%, 65%, 70%, 75%, 80%, 85%, 90%, 95%, or 100% of the number of the same type of atoms in comparison to a substituted compound or polyunsaturated lipid with no heavy atoms of the same type (e.g. hydrogen would be the “same type” as deuterium). In some embodiments, the isotopic load is at least 5%, 10%, 15%, 20%, 25%, 30%, 35%, 40%, 45%, 50%, 65%, 60%, 65%, 70%, 75%, 80%, 85%, 90%, 95%, or 100%. Unintended side effects are expected to be reduced where there is high isotopic purity in a substituted compound or polyunsaturated lipid composition but low isotopic load in a given molecule. For example, the metabolic pathways will likely be less affected by using a substituted compound or polyunsaturated lipid composition with high isotopic purity but low isotopic load.

One will readily appreciate that when one of the two hydrogen s of a methyl ene group is replaced with a deuterium atom, the resultant compound may possess a stereo center. In some embodiments, it may be desirable to use racemic compounds. In other embodiments, it may be desirable to use enantiomerically pure compounds. In additional embodiments, it may be desirable to use diastereomerically pure compounds. In some embodiments, it may be desirable to use mixtures of compounds having enantiomeric excesses and/or diastereomeric excesses of about 5%, 10%, 15%, 20%, 25%, 30%, 35%, 40%, 45%, 50%, 65%, 60%, 65%, 70%, 75%, 80%, 85%, 90%, 95%, or 100%. In other embodiments, the enantiomeric excesses and/or diastereomeric excesses is at least 5%, 10%, 15%, 20%, 25%, 30%, 35%, 40%, 45%, 50%, 65%, 60%, 65%, 70%, 75%, 80%, 85%, 90%, 95%, or 100%. In some embodiments, it may be preferable to utilize stereochemically pure enantiomers and/or diastereomers of embodiments—such as when contact with chiral molecules is being targeted for attenuating oxidative damage. However, in many circumstances, non-chiral molecules are being targeted for attenuating oxidative damage. In such circumstances, embodiments may be utilized without concern for their stereochemical purity. Moreover, in some embodiments, mixtures of enantiomers and diastereomers may be used even when the compounds are targeting chiral molecules for attenuating oxidative damage.

In some aspects, an isotopically modified polyunsaturated lipid imparts an amount of heavy atoms in a particular tissue. Thus, in some aspects, the amount of heavy molecules will be a particular percentage of the same type of molecules in a tissue. For example, the number of heavy molecules may be about 1%-100% of the total amount of the same type of molecules in a tissue. In some aspects, 10-50% of the molecules are substituted with the same type of heavy molecules.

In some embodiments, the polyunsaturated lipid is deuterated at one or more bis-allylic positions. One example of a polyunsaturated lipid is an essential PUFAs that is isotopically modified at bis-allylic positions as shown below in Formula (3), whereas R¹ is —O-alkyl, —OH, amine, —SH, —S-alkyl, or —O-cation (e.g., cation is Na⁺ or K⁺); m is 1 to 10; n is 1 to 5; R is H or alkyl (e.g., C₃H₇). The bis-allylic positions, in addition to deuteration, can be further reinforced by carbon-13, each at levels of isotope abundance above the naturally-occurring abundance level. At each bis-allylic position in Formula (3), one or both Y⁵, Y² atoms in each n unit are independently deuterium atoms. In some embodiments, n is 1, 2, 3, or 4. In some embodiments, m is 1, 2, 3, or 4.

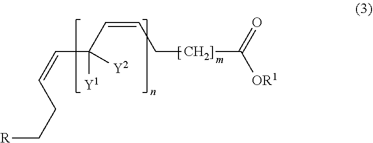

Exact structures of compounds illustrated above are shown below that provide for both isotope reinforced n-3 (omega-3) and n-6 (omega-6) essential polyunsaturated fatty acids, and the PUFAs made from them biochemically by desaturation/elongation. Any one of these compounds may be used to slow oxidation. In the following compounds, the PUFAs are isotopically reinforced at oxidation sensitive sites and/or sites that may become oxidation sensitive upon biochemical desaturation/elongation. R¹ may be H, alkyl, or cation (e.g., Na+ or K+); R² may be H or D; * represents either ¹²C or ¹³C.

Deuterated linoleic acids may include:

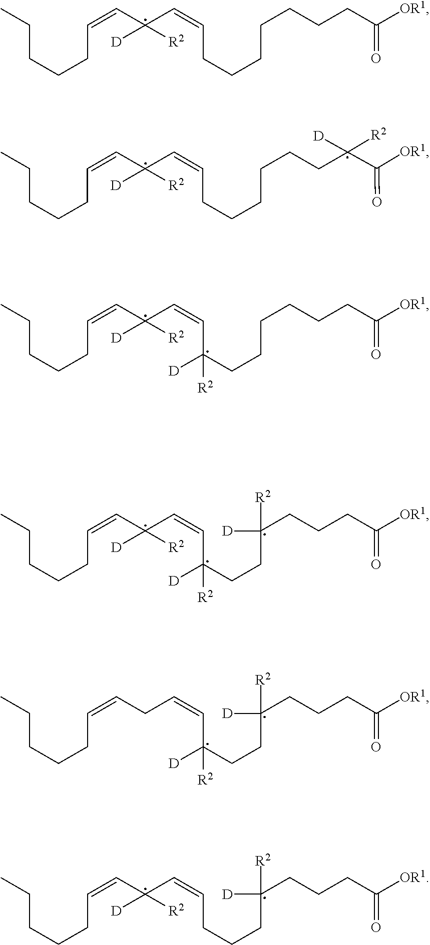

The per-deuterated linoleic acid below may be produced by microbiological methods, for example by growing in media containing deuterium and/or carbon-13.

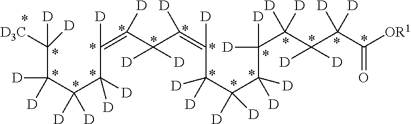

Deuterated arachidonic acids may include:

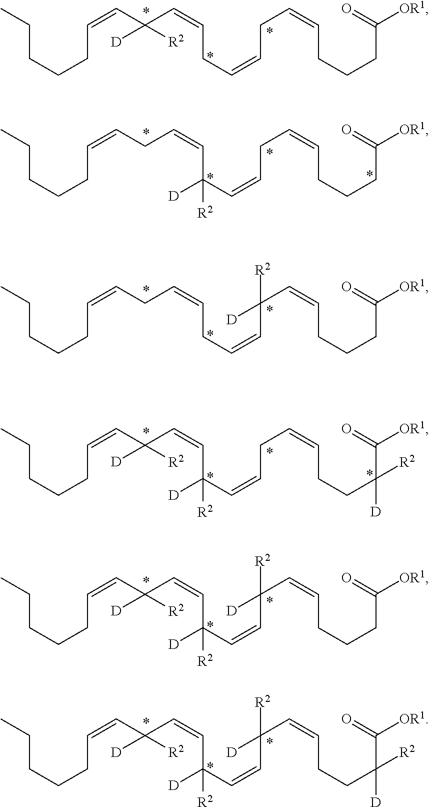

The per-deuterated arachidonic acid below may be produced by microbiological methods, such as by growing in media containing deuterium and/or carbon-13.

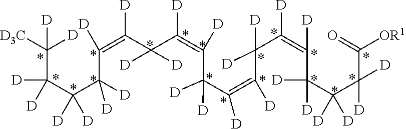

Deuterated linolenic acids may include:

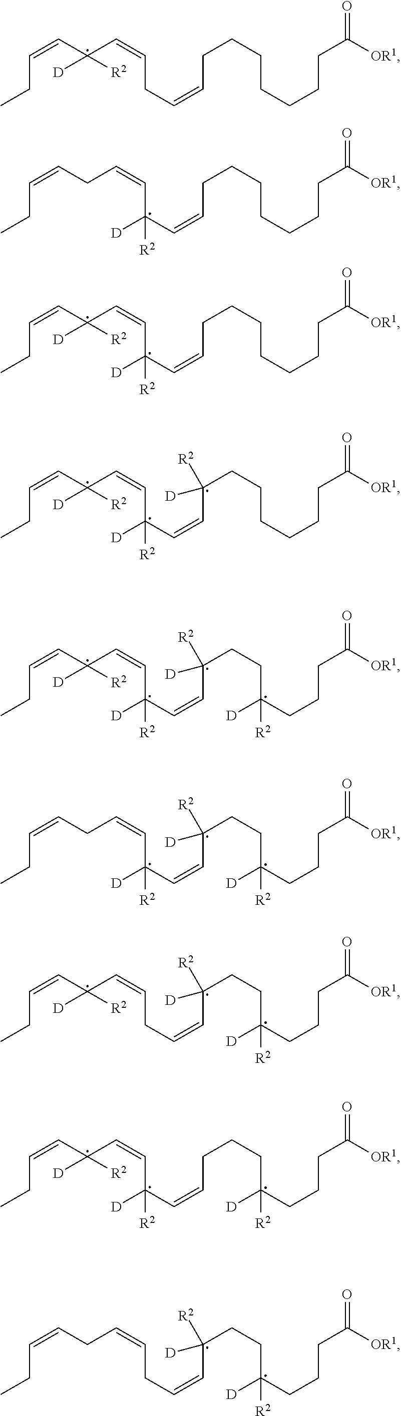

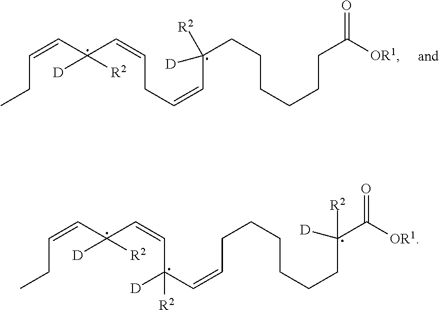

Per-deuterated linolenic acid below may be produced by microbiological methods, such as growing in media containing deuterium and/or carbon-13.

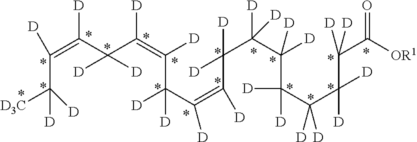

Deuterated polyunsaturated fatty acid and ester may also include:

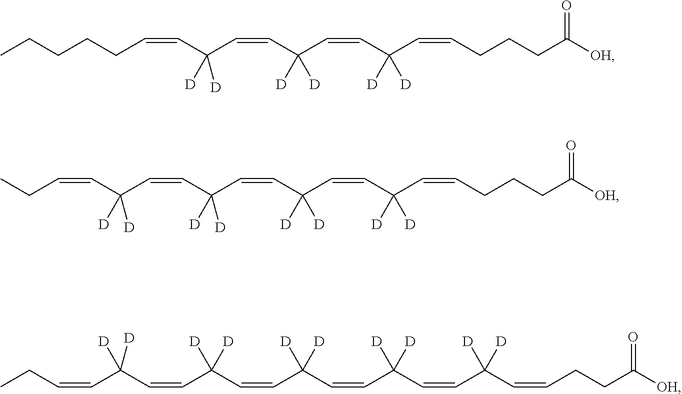 [SMILES: [2H]C([2H])(/C=C\CCCCC)/C=C\C([2H])([2H])/C=C\C([2H])([2H])/C=C\CCCC(=O)O ; [2H]C([2H])(/C=C\CC)/C=C\C([2H])([2H])/C=C\C([2H])([2H])/C=C\C([2H])([2H])/C=C\CCCC(=O)O ; [2H]C([2H])(/C=C\CC)/C=C\C([2H])([2H])/C=C\C([2H])([2H])/C=C\C([2H])([2H])/C=C\C([2H])([2H])/C=C\CCC(=O)O]

and ester thereof.

In a further embodiment, oxidation-prone bis-allylic sites of substituted compounds as described herein (e.g., PUFAs) can be protected against hydrogen abstraction by moving bis-allylic hydrogen-activating double bonds further apart, thus eliminating the bis-allylic positions while retaining certain PUFA functionality as shown below. These PUFA mimetics have no bis-allylic positions.

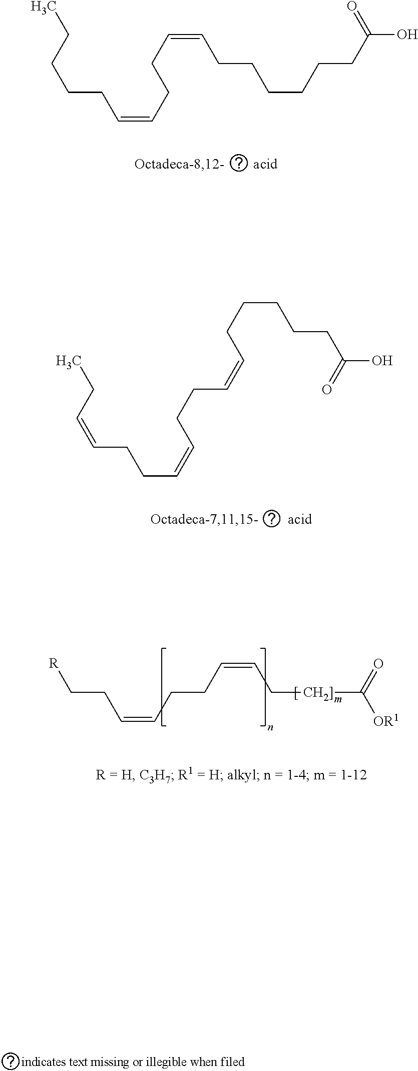

In a further embodiment, oxidation-prone bis-allylic sites of substituted compounds as described herein (e.g., PUFAs) can be protected against hydrogen abstraction by incorporating heteroatoms with valence II (e.g., S, O), thus eliminating the bis-allylic hydrogens as shown below. These PUFA mimetics also have no bis-allylic hydrogens.

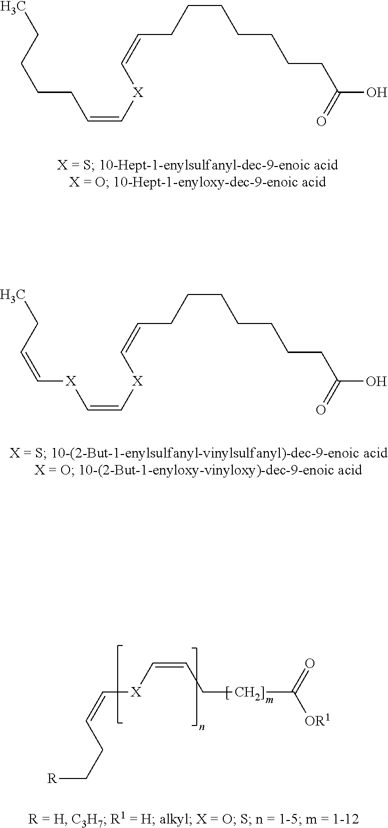

In a further embodiment, PUFA mimetics, i.e. compounds structurally similar to natural PUFAs but more resistant to oxidation because of the structural differences, can be employed for the above mentioned purposes. Oxidation-prone bis-allylic sites of PUFAs can be protected against hydrogen abstraction by di-methylation or halogenation as shown below. The hydrogen atoms on the methyl groups may optionally be halogens, such as fluorine, or deuterium. These PUFA mimetics are demethylated at bis-allylic sites.

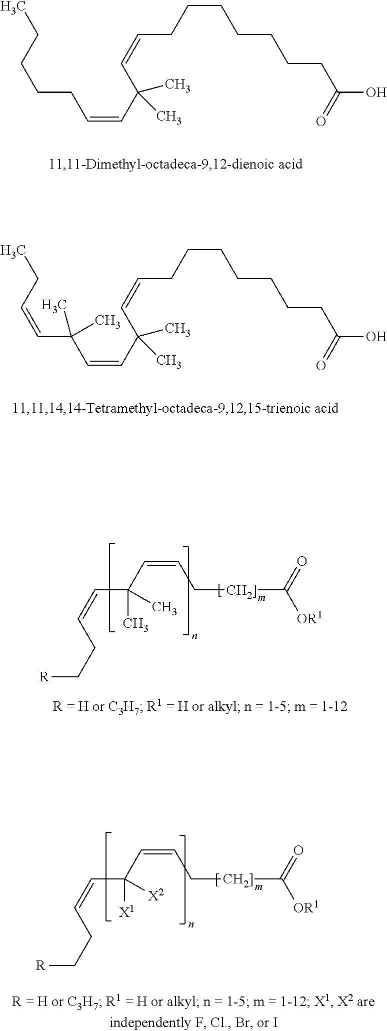

In a further embodiment, oxidation-prone bis-allylic sites of PUFAs can be protected against hydrogen abstraction by alkylation as shown below. These PUFA mimetics are dialkylated at bis-allylic sites.

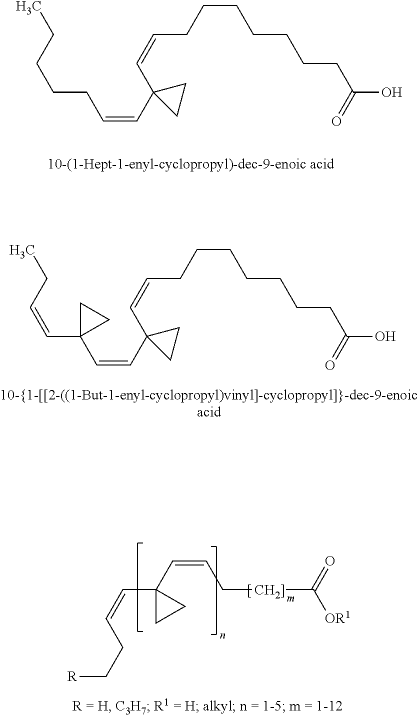

In a further embodiment, cyclopropyl groups can be used instead of double bonds, thus rendering the acids certain functions while eliminating the bis-allylic sites as shown below. These PUFA mimetics have cyclopropyl groups instead of double bonds.

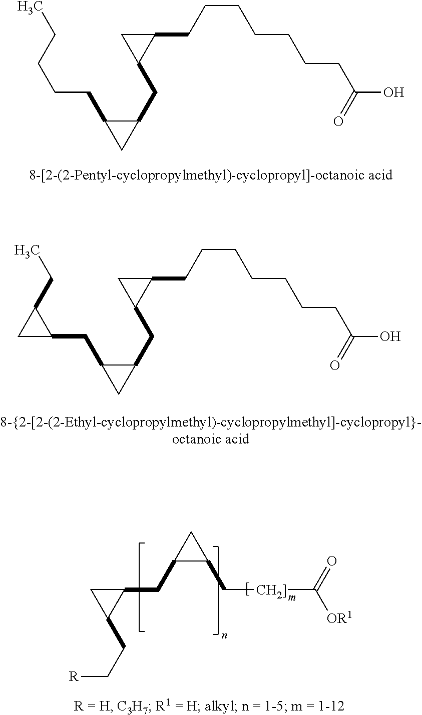

In a further embodiment, 1,2-substituted cyclobutyl groups in appropriate conformations can be used instead of double bonds, thus rendering the acids certain functions while eliminating the bis-allylic sites as shown below. These PUFA mimetics have 1,2-cyclobutyl groups instead of double bonds.

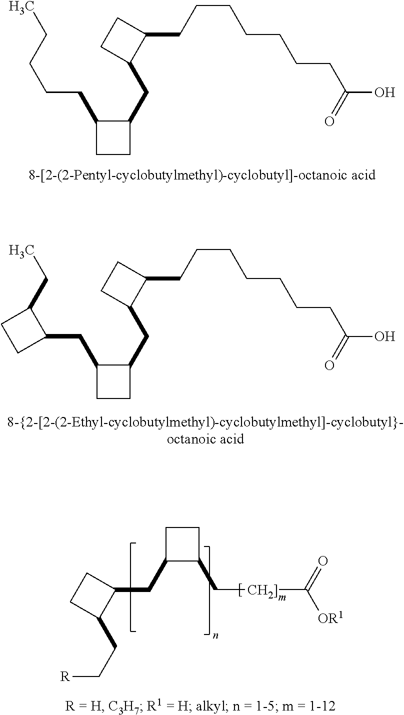

In a modification of the previous embodiment of mimetics with 1,2-cyclobutyl groups instead of double bonds, 1,3-substituted cyclobutyl groups in appropriate conformations can be used instead of double bonds, thus rendering the acids certain functions while eliminating the bis-allylic sites. The following PUFA mimetics have 1,3-cyclobutyl groups instead of double bonds.

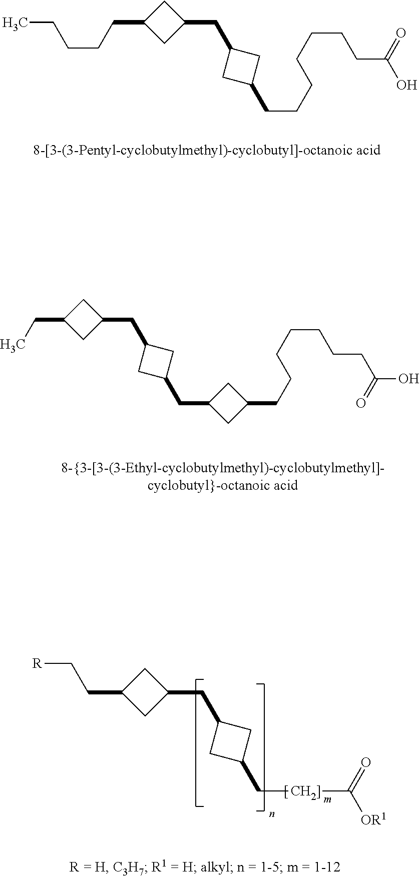

Bioisosteres are substituents or groups with similar physical or chemical properties which produce broadly similar biological properties to a chemical compound. For example, well known isosteres and/or bioisosteres for hydrogen include halogens such as fluorine; isosteres and/or bioisosteres of alkenes include alkynes, phenyl rings, cyclopropyl rings, cyclobutyl rings, cyclopentyl rings, cyclohexyl rings, thioethers, and the like; isosteres and/or bioisosteres of carbonyls include sulfoxides, sulfones, thiocarbonyls, and the like; isosteres and/or bioisosteres of esters include amides, sulfonic acid esters, sulfonamides, sulfinyl acid esters, sulfinylamindes, and the like. Consequently, PUFA mimetics also include compounds having isosteric and/or bioisosteric functional groups.

In some embodiments, PUFAs and/or PUFA mimetics are formulated as a pro-drug for use in the various methods described herein. A pro-drug is a pharmacological substance that may itself have biological activity, but upon administration the pro-drug is metabolized into a form that also exerts biological activity. Many different types of pro-drugs are known and they can be classified into two major types based upon their cellular sites of metabolism. Type I pro-drugs are those that are metabolized intracellularly, while Type II are those that are metabolized extracellularly. It is well-known that carboxylic acids may be converted to esters and various other functional groups to enhance pharmacokinetics such as absorption, distribution, metabolism, and excretion. Esters are a well-known pro-drag form of carboxylic acids formed by the condensation of an alcohol (or its chemical equivalent) with a carboxylic acid (or its chemical equivalent). In some embodiments, alcohols (or their chemical equivalent) for incorporation into pro-drugs of PUFAs include pharmaceutically acceptable alcohols or chemicals that upon metabolism yield pharmaceutically acceptable alcohols. Such alcohols include, but are not limited to, propylene glycol, ethanol, isopropanol, 2-(2-ethoxyethoxy)ethanol (Transcutol®, Gattefosse, Westwood, N.J. 07675), benzyl alcohol, glycerol, polyethylene glycol 200, polyethylene glycol 300, or polyethylene glycol 400; polyoxyethylene castor oil derivatives (for example, polyoxyethyleneglyceroltriricinoleate or polyoxyl 35 castor oil (Cremophor®EL, BASF Corp.), polyoxyethyleneglycerol oxystearate (Cremophor®RH 40 (polyethyleneglycol 40 hydrogenated castor oil) or Cremophor®RH 60 (polyethyleneglycol 60 hydrogenated castor oil), BASF Corp.)); saturated polyglycolized glycerides (for example, Gelucire® 35/10, Gelucire® 44/14, Gelucire®46/07, Gelucire® 50/13 or Gelucire® 53/10, available from Gattefosse, Westwood, N.J. 07675); polyoxyethylene alkyl ethers (for example, cetomacrogol 1000); polyoxyethylene stearates (for example, PEG-6 stearate, PEG-8 stearate, polyoxyl 40 stearate NF, polyoxyethyl 50 stearate NF, PEG-12 stearate, PEG-20 stearate, PEG-100 stearate, PEG-12 distearate, PEG-32 distearate, or PEG-150 distearate); ethyl oleate, isopropyl palmitate, isopropyl myristate; dimethyl isosorbide; N-methylpyrrolidinone; paraffin; cholesterol; lecithin; suppository bases; pharmaceutically acceptable waxes (for example, carnauba wax, yellow wax, white wax, microcrystalline wax, or emulsifying wax); pharmaceutically acceptable silicon fluids; sorbitan fatty acid esters (including sorbitan laurate, sorbitan oleate, sorbitan palmitate, or sorbitan stearate); pharmaceutically acceptable saturated fats or pharmaceutically acceptable saturated oils (for example, hydrogenated castor oil (glyceryl-tris-12-hydroxystearate), cetyl esters wax (a mixture of primarily C₁₄-C₁₈ saturated esters of CM-CIS saturated fatty acids having a melting range of about 43°-47° C.), or glyceryl monostearate) and combinations thereof.

In some embodiments, the fatty acid pro-drug is represented by the ester P-B, wherein the radical P is a PUFA and the radical B is a biologically acceptable molecule. Thus, cleavage of the ester P-B affords a PUFA and a biologically acceptable molecule. Such cleavage may be induced by acids, bases, oxidizing agents, and/or reducing agents. Examples of biologically acceptable molecules include, but are not limited to, nutritional materials, peptides, amino acids, proteins, carbohydrates (including monosaccharides, disaccharides, polysaccharides, glycosaminoglycans, and oligosaccharides), nucleotides, nucleosides, lipids (including mono-, di- and tri-substituted glycerols, glycerophospholipids, sphingolipids, and steroids) and combinations thereof.

In some embodiments, alcohols (or their chemical equivalent) for incorporation into pro-drugs of PUFAs include polyalcohols such as diols, triols, tetra-ols, penta-ols, etc. Examples of polyalcohols include ethylene glycol, propylene glycol, 1,3-butylene glycol, polyethylene glycol, methylpropanediol, ethoxydiglycol, hexylene glycol, dipropylene glycol glycerol, and carbohydrates. Esters formed from polyalcohols and PUFAs may be mono-esters, di-esters, tri-esters, etc. In some embodiments, multiply esterified polyalcohols are esterified with the same PUFAs. In other embodiments, multiply esterified polyalcohols are esterified with different PUFAs. In some embodiments, the different PUFAs are stabilized in the same manner. In other embodiments, the different PUFAs are stabilized in different manners (such as deuterium substitution in one PUFA and ¹³C substitution in another PUFA). In some embodiments, one or more PUFAs is an omega-3 fatty acid and one or more PUFAs is an omega-6 fatty acid.

It is also contemplated that it may be useful to formulate PUFAs and/or PUFA mimetics and/or PUFA pro-drugs as salts for use, e.g., as pharmaceutically acceptable salts. For example, the use of salt formation as a means of tailoring the properties of pharmaceutical compounds is well known. See Stahl et al., Handbook of pharmaceutical salts: Properties, selection and use (2002) Weinheim/Zurich: Wiley-VCH/VHCA; Gould, Salt selection for basic drugs, Int. J. Pharm. (1986), 33:201-217. Salt formation can be used to increase or decrease solubility, to improve stability or toxicity, and to reduce hygroscopicity of a drug product.

Formulation of PUFAs and/or PUFA esters and/or PUFA mimetics and/or PUFA pro-drugs as salts can include any PUFA described herein.

It may be unnecessary to substitute all isotopically unmodified PUFAs, such as non-deuterated PUFAs, with isotopically modified PUFAs such as deuterated PUFAs. In some embodiments, is preferable to have sufficient isotopically modified PUFAs such as D-PUFAs in the membrane to prevent unmodified PUFAs such as H-PUFAs from sustaining a chain reaction of self-oxidation. During self-oxidation, when one PUFA oxidizes, and there is a non-oxidized PUFA in the vicinity, the non-oxidized PUFA can get oxidized by the oxidized PUFA. This may also be referred to as autoxidation. In some instances, if there is a tow concentration, for example “dilute” H-PUFAs in the membrane with D-PUFAs, this oxidation cycle may be broken due to the distance separating H-PUFAs. In some embodiments, the isotopically modified PUFAs is present in a sufficient amount to break an autoxidation chain reaction. To break the autoxidation chain reaction, for example, effective amounts of isotopically modified PUFAs may be 1-60%, 5-50%, or 15-35% of the total molecules of the same type in the membrane.

### Pharmaceutical Compositions

Some embodiments include pharmaceutical compositions comprising: (a) a safe and therapeutically effective amount of a substituted compound described herein; and (b) a pharmaceutically acceptable carrier, diluent, excipient or combination thereof. In some embodiments, the substituted compound is an isotopically modified polyunsaturated acid (PUFA) or an ester, thioester, amide, or other prodrug thereof, or combinations thereof. In some further embodiment, the isotopically modified PUFA is 11,11-D2-linoleic acid or an ester thereof. In one particular embodiment, the isotopically modified PUFA is 11,11-D2-linoleic acid ethyl ester.

The compounds useful as described above can be formulated into pharmaceutical compositions for use in treatment of various conditions or disorders. Standard pharmaceutical formulation techniques are used, such as those disclosed in Remington's The Science and Practice of Pharmacy, 21st Ed., Lippincott Williams & Wilkins (2005), incorporated by reference in its entirety.

In addition to the selected compound useful as described above, some embodiments include compositions containing a pharmaceutically-acceptable earner. The term “pharmaceutically-acceptable carrier”, as used herein, means one or more compatible solid or liquid filler diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy of the composition under ordinary use situations. Pharmaceutically-acceptable carriers must, of course, be of sufficiently high purity and sufficiently low toxicity to render them suitable for administration preferably to an animal, preferably mammal being treated.

Pharmaceutically-acceptable carriers include, for example, solid or liquid fillers, diluents, hydrotropies, surface-active agents, and encapsulating substances. Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives; pyrogen-free water; isotonic saline; and phosphate buffer solutions.

Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carriers suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Per-oral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives or supplements. For example, the pharmaceutical composition is administered concomitantly with one or more antioxidants. In some embodiments, the antioxidant is selected from the group consisting of Coenzyme Q, idebenone, mitoquinone, mitoquinol, vitamin E, and vitamin C, and combinations thereof. In some such embodiments, at least one antioxidant may be taken concurrently, prior to, or subsequent to the administration of the substituted compound described herein. In some embodiments, the antioxidant and the substituted compounds be in a single dosage form. In some embodiments, the single dosage form is selected from the group consisting of a pill, a tablet, and a capsule.

It will be understood by those of skill in the art that numerous and various modifications can be made without departing from the spirit of the present disclosure. Therefore, it should be clearly understood that the embodiments disclosed herein are illustrative only and are not intended to limit the scope of the present invention. Any reference referred to herein is incorporated by reference for the material discussed herein, and in its entirety.

### EXAMPLES

### Example 1

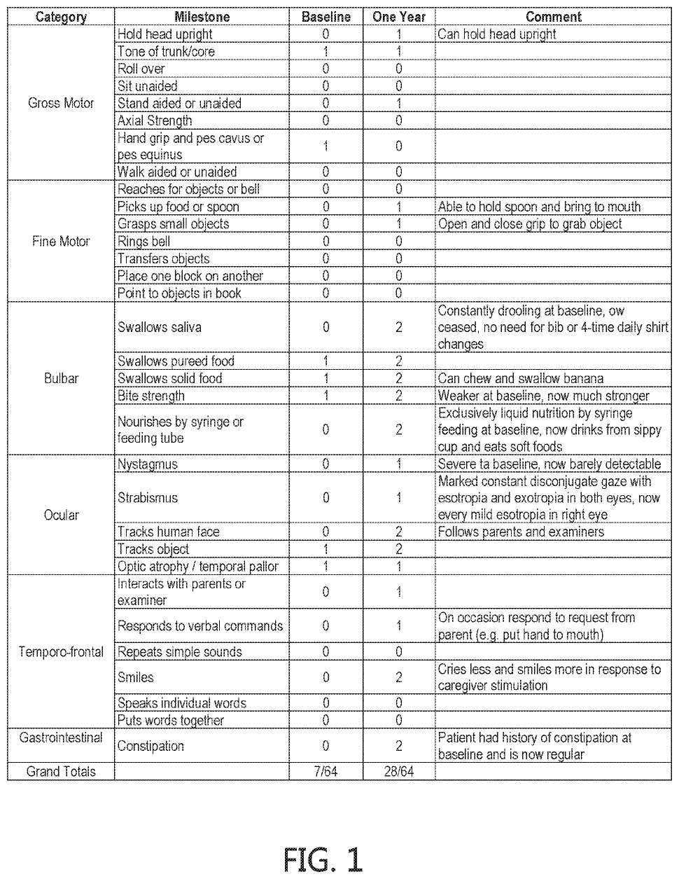

A study in the fatal neurodegenerative disease, Infantile Neuroaxonal Dystrophy (INAD) was initiated in March 2017. At study onset, the subject, a 2.5 year old female, with a genetic mutation in both copies of the PLA2G6 gene, was mostly unresponsive to stimuli and unable to perform virtually any activity. All normal development milestones previously acquired had been lost. Prognosis was that a feeding tube would be required in the near term progression of the disease. The patient received dosing of a D-PUFA (11,11-D2-linoleic acid), 1.8 g twice a day, for a six month trial under an Expanded Access (Compassionate Use) protocol. As she was unable to swallow a pill, soft gels containing the D-PUFA were placed in warm liquid (water or milk), pierced with a fork, and the active oil pressed into the liquid, which was then soaked into food or a cookie, and administered. Starting within a month of dosing and continuing until the current one year anniversary of dosing, the patient has improved. FIG. 1 shows a detailed list of development milestones lost by the subject prior to drug treatment, and the observations of the treating physicians versus the baseline assessment at the start of trial. These results were recorded in videotaped exams at baseline, 1 month, 3, 6, 9 and 12 months.

FIG. 1 summarizes the baseline and one year treatment status of the patient (degree of impairment: (0) for severely impaired, (+1) for moderately impaired, (+2) for mildly impaired or no impairment).

Within the first week, chronic constipation resolved. After 2 months on drug, she regained sufficient bulbar function to terminate syringe feeding of liquids and returned to drinking from a child's sippy cup.

After 4 months on drug, there were no drug related adverse events of any sort reported. One month and three month plasma level of deuterated polyunsaturated fatty acid was at steady state at approximately 44% total (dietary plus modified) linoleic acid, indicating excellent uptake and absorption of a therapeutic level of drug (>˜10-15%). Deuterated arachidonic acid was observed as present in the plasma at 0.6% and 3.0% of total arachidonic acid at month one and three months; respectively. In addition, clinical examination at 3 months showed no progression of disease since the start of the trial. Importantly, both clinical examination video, medical notes, and caregiver reports revealed the following observations: 11,11-D2-linoleic acid was safe and well-tolerated at high dose in a 2 year old; the subject's disease did not progress (n=1); parents noted improvement as follows at 4 months including regained ability to grasp spoon and hold; regained ability to swallow using sipper cup (vs. syringe feeding); can eat (chew and swallow) food such as banana; salivation down 99%, essentially back to normal; better muscle strength (grasping and lifting rattle); newly responsive to verbal requests during therapy; and constipation resolved, subject completely regular (constipation is a symptom of PLA2G6 diseases).

After 6 months, she improved in qualitative measures captured in videotaped therapy sessions or exams, including eye tracking, responsiveness to verbal commands, head control, lifting, and reaching and grasping for her spoon (a lost skill). INAD is a strictly progressive disease, only worsening with time. Stabilization of regressions and recovery of lost milestones indicated effectiveness of the test treatment in a single, severely affected INAD patient.

Stabilization of progression of lost development milestones is a major advance in therapy for INAD. In view of the clinical study results, these clear reversals indicate that substituted compounds as described herein are effective for treating a subject having, or at risk for, a disease or condition associated with an impaired Phospholipase A2 Group VI (PLA2G6) activity, and particularly the stabilized PUFA (11,11-D2-linoleic acid), in patients with classical INAD.

### Example 2

In this example, a case study of using 11,11-D2-linoleic acid ethyl ester to treat a single patient with late onset Tay-Sachs disease (LOTS) and the study results was reported.

Design/Methods: 11,11-D2-linoleic acid ethyl ester was administered to the patient at 2.7 g (BID) and periodic repeat assessments including baseline measurement of PK, activities of daily living (ADL), 25 foot walk time (25FWT), and 6 minute walk distance (6MWD) were made. In particular, ADLs were measured individually on a scale of 0-5 across a 12 element panel representing speech, strength, coordination, etc.

Results: 11,11-D2-linoleic acid (D-LA) was elongated to 13,13-D2-arachidonic acid (D-AA) and both deuterated PUFAs achieve significant plasma levels and red blood cell (RBC) membrane incorporation within 1 month of administration. Improvements in ADL, 25FWT, and 6MWD have also been seen. No major toxicities have been seen (Table 1).

TABLE 1

| | D-LA(%) | | | | | |
|---|---|---|---|---|---|---|
| | D-LA + H-LA | | Composite | # of ADLs | 6MWD | 25FWT |
| Time | Plasma | RBC | ADL Score | improved | (M) | (Sec) |
| Baseline | 0 | 0 | 0 | n/a | 112 | 12.7 |
| 30 d | 36 | 28 | +6 | 6 | n/a | 12.1 |
| 120 d | n/a | n/a | +9 | 8 | 129 | 11.2 |

Conclusion: Early efficacy signs of inhibition of disease progression and some regression were seen. Furthermore, 11,11-D2-linoleic acid ethyl ester was well tolerated with no toxicities reported.

What is claimed is:

1. A method of treating a subject having, or at risk for, a disease or condition associated with an impaired Phospholipase A2 Group VI (PLA2G6) activity, comprising:
selecting a subject having, or at risk for, a disease or condition associated with an impaired Phospholipase A2 Group VI (PLA2G6) activity; and
administering to the subject an effective amount of a substituted compound selected from the group consisting of a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drug, and combinations thereof, wherein the substituted compound comprises at least one substituent that reduces oxidation of the substituted compound.

2. The method of claim 1, wherein the subject has infantile neuroaxonal dystrophy (INAD) or PLA2G6 associated neurodegeneration (PLAN).

3. A method of treating a subject having, or at risk for, a disease or condition associated with a lysosomal storage disease (LSD) and/or neuronal ceroid lipofuscinosis (NCL) disease, comprising:
selecting a subject having, or at risk for, a disease or condition associated with a lysosomal storage disease or neuronal ceroid lipofuscinosis; and
administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drag, or combinations thereof, wherein the substituted compound comprises at least one substituent that reduces oxidation of the substituted compound.

4. The method of claim 3, wherein the subject has Tay-Sachs, Gaucher disease, Sandhoff disease, or Niemann-Pick disease.

5. The method of claim 4, wherein the subject has Tay-Sachs disease.

6. A method of treating a subject having, or at risk for, a sleeping disorder, comprising:
selecting a subject having, or at risk for, a sleeping disorder; and
administering to the subject an effective amount of a substituted compound selected from a polyunsaturated fatty acid, a polyunsaturated fatty acid ester, a polyunsaturated fatty acid thioester, a fatty acid amide, a polyunsaturated fatty acid mimetic, a polyunsaturated fatty acid pro-drag, or combinations thereof, wherein the substituted compound comprises at least one substituent that reduces oxidation of the substituted compound.

7. The method of claim 6, wherein the subject has acute or chronic dyssomnia, or obstructive sleep apnoea syndrome.

8. The method of any one of claims 1 to 7, wherein the substituted compound comprises one or more isotope atoms, and wherein the amount of the isotope atoms is significantly above the naturally-occurring abundance level of the isotope atom.

9. The method of claim 8, wherein the isotope atoms are deuterium, ¹³C, or a combination thereof.

10. The method of any one of claims 1 to 9, wherein the administering comprises repeated administration.

11. The method of any one of claims 1 to 10, wherein the subject has or is at risk for at least one of neuropathy or a neurodegenerative disease and the amount of the substituted compound is effective to prevent, ameliorate or inhibit the progression of neuropathy or the neurodegenerative disease.

12. The method of any one of claims 1 to 11, wherein the substituted compound is an isotopically modified polyunsaturated fatty acid, or an ester, an amide, a thioester, or a prodrug thereof.

13. The method of claim 12, wherein the substituted compound is a ω-3 fatty acid, a ω-6 fatty acid, a ω-3 fatty acid ester, a ω-6 fatty acid ester, a ω-3 fatty acid amide, a ω-6 fatty acid amide, a ω-3 fatty acid thioester, or a ω-6 fatty acid thioester, or combinations thereof.

14. The method of any one of claims 1 to 13, wherein the polyunsaturated fatty acid ester is selected from a triglyceride, a diglyceride, a monoglyceride, or an alkyl ester.

15. The method of any one of claims 1 to 14, wherein the substituted compound has the structure of formula (1): 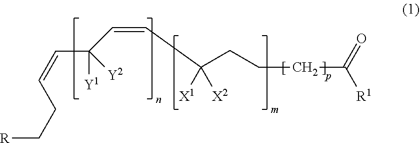
wherein R is H or C₃H₇;
R¹ is OH, O-alkyl, amine, S-alkyl, or O-cation;
each Y¹ and Y² is independently H or D;
each X¹ and X² is independently H or D, wherein at least one of Y¹, Y², X¹ and X² is D;
m is 0, 1, 2, 3, 4, 5, 6, 7, 8, 9 or 10;
n is 1, 2, 3, 4, or 5; and
p is 0, 1, 2, 3, 4, 5, 6, 7, 8, 9 or 10.

16. The method of any one of claims 1 to 15, wherein the substituted compound is a deuterated linoleic acid, a deuterated linolenic acid, a deuterated arachidonic acid, a deuterated eicosapentaenoic acid, a deuterated docosahexaenoic acid, or an ester, an amide, or a thioester thereof.

17. The method of claim 16, wherein the amount of deuterium in the substituted compound is significantly above the naturally-occurring abundance level of the deuterium.

18. The method of claim 17, wherein the substituted compound is deuterated at one or more bis-allyl positions.

19. The method of claim 17, wherein the substituted compound is selected from the group consisting of 11,11-D2-linolenic acid; 14,14-D2-linolenic acid; 11,11,14,14-D4-linolenic acid; 11,11-D2-linoleic acid; 7,7-D2-arachidonic acid; 10,10-D2-arachidonic acid; 13,13-D2-arachidonic acid; 7,7,10,10-D4-arachidonic acid; 7,7,13,13-D4-arachidonic acid; 10,10,13,13-D4-arachidonic acid; 7,7,10,10,13,13-D6-arachidonic acid; 7,7,10,10,13,13,16,16-D8-eicosapentaenoic acid; 6,6,9,9,12,12,15,15,18,18-D10-docosahexaenoic acid; an ester of any of the foregoing, and combinations thereof.

20. The method of any one of claims 1 to 19, wherein the ester is an ethyl ester.

21. The method of claim 20, wherein the substituted compound is 11,11-D2-linoleic acid ethyl ester.

22. The method of claim 20, wherein the substituted compound is 11,11,14,14-D4-linolenic acid ethyl ester.

23. The method of any one of claims 1 to 22, wherein the subject also ingests at least one of an unsubstituted polyunsaturated fatty acid and an unsubstituted polyunsaturated fatty acid ester.

24. The method of claim 23, wherein the amount of the substituted compound is about 5% or greater than the total amount of the polyunsaturated fatty acids and polyunsaturated fatty acid esters administered or delivered to the subject.

25. The method of claim 23, wherein the amount of the substituted compound is equal to or less than about 1% of the total amount of the polyunsaturated fatty acids and polyunsaturated fatty acid esters administered or delivered to the subject.

26. The method of any one of claims 1 to 25, wherein the amount of the substituted compound administered is from about 10 mg/kg to about 200 mg/kg.

27. The method of any one of claim 26, wherein the amount of the substituted compound administered is from about 20 mg/kg to about 100 mg/kg.

28. The method of any one of claims 1 to 27, wherein the amount of the substituted compound administered is from about 1 g to about 10 g.

29. The method of claim 28, wherein the amount of the substituted compound administered is from about 2 g or about 5 g.

30. The method of any one of claims 1 to 29, wherein the substituted compound is administered once per day.

31. The method of any one of claims 1 to 29, wherein the substituted compound is administered two or more times per day.

32. The method of any one of claims 1 to 31, wherein the amount of the substituted compound administered is from about 1 g to about 20 g per day.

33. The method of claim 32, wherein the amount of substituted compound administered is from about 2 g to about 10 g per day.

34. The method of any one of claims 1 to 33, wherein the substituted compound is administered for at least 2, 3, or 4 weeks.

35. The method of any one of claims 1 to 34, wherein the substituted compound is co-administered to the subject with at least one antioxidant.

36. The method of claim 35, wherein the antioxidant is selected from the group consisting of Coenzyme Q, idebenone, mitoquinone, mitoquinol, plastoquinone, resveratrol, vitamin E, and vitamin C, and combinations thereof.

37. The method of any one of claims 1, 2 and 8 to 36, wherein the amount of the substituted compound administered is effective to alleviate one or more symptoms of the disease or condition associated with the impaired Phospholipase A2 Group VI (PLA2G6) activity.

38. The method of claim 37, wherein the symptom associated with the impaired PLA2G6 activity is selected from the group consisting of hypotonia, nystagmus, strabismus, psychomotor regression, and low spontaneous motor activity, and combinations thereof.

39. The method of any one of claims 3 to 5 and 8 to 36, wherein the amount of the substituted compound administered to the subject is effective to alleviate one or more symptoms associated with LSD and/or NCL.

40. The method of claim 39, wherein the symptom associated with LSD and/or NCL is selected from the group consisting of difficulties with physical movement, joint stiffness and pain, seizures, dementia, mental retardation, high fatality, problems with vision, problems with hearing, and problems with bulbar function, and combinations thereof.

41. The method of any one of claims 6 to 36, wherein the amount of the substituted compound administered to the subject is effective to alleviate one or more symptoms or side effects associated with a sleeping disorder or insufficient sleep.

42. The method of any one of claims 1 to 41, wherein the amount of the substituted compound administered is effective to improve a muscle function of the subject.

43. The method of claim 42, wherein the muscle function is selected from the group consisting of eye tracking, control, lifting, fine motor skill, and muscle strength, and combinations thereof.

44. The method of any one of claims 1 to 43, wherein the amount of the substituted compound administered is effective to improve a neural function of the subject.

45. The method of claim 44, wherein the neural function is selected from responsiveness to verbal commands, bulbar function, and verbal cognition, and combinations thereof.
